(19) United States
(12) Patent Application Publication
(10) Pub. No.: US 2022/0288218 A1
(43) Pub. Date: Sep. 15, 2022

(54) EXONS 45-55 SKIPPING USING MUTATION-TAILORED COCKTAILS OF ANTISENSE MORPHOLINOS IN THE DMD GENE

(71) Applicant: The Governors of the University of Alberta, Edmonton (CA)

(72) Inventors: Toshifumi YOKOTA, Edmonton (CA); Yusuke ECHIGOYA, Fujisawa (JP); Kenji Rowel Quintana LIM, Edmonton (CA)

(21) Appl. No.: 17/625,545

(22) PCT Filed: Jul. 9, 2020
(86) PCT No.: PCT/CA2020/050948

§ 371 Date: Jan. 7, 2022

Related U.S. Application Data
(60) Provisional application No. 62/871,797, filed on Jul. 9, 2019.

(51) Int. Cl. A61K 47/64; A61K 31/7088; A61P 21/00; A61K 9/00

(52) U.S. Cl. CPC A61K 47/645; A61K 31/7088; A61P 21/00; A61K 9/0019

(57) ABSTRACT

Described herein is/are a therapeutic antisense oligonucleotide(s) which binds to exons 45 to 55 of the human dystrophin pre-mRNA to induce exon skipping, and conjugates and compositions thereof for the treatment of DMD.

### CROSS REFERENCE TO RELATED APPLICATION

This application claims priority to U.S. Provisional Patent Application 62/871,797, filed Jul. 9, 2019, the entire contents of which is hereby incorportated by reference.

### FIELD

The present disclosure relates generally to a therapeutic antisense oligonucleotide(s) which binds to exons 45 to 55 of the human dystrophin pre-mRNA to induce exon skipping, and conjugates and compositions thereof for the treatment of DMD.

### BACKGROUND

Duchenne muscular dystrophy (DMD), a lethal X-linked recessive neuromuscular disorder, is caused by mutations in the dystrophin (DMD) gene and the absence of dystrophin for maintaining muscle membrane integrity.¹ Although the DMD gene is the largest known in humans consisting of 79 exons in 2.4 Mb, there exists a mutational hotspot ranging from exon 43 to 55.² Deletions are the most frequent mutations to occur and account for approx. 68% of cases.³ Of them, severe DMD results from mostly out-of-frame deletions that do not allow for the production of dystrophin. In contrast, in-frame deletions, which permits the production of internally-truncated dystrophins, mostly give rise to the mild counterpart, Becker muscular dystrophy (BMD).⁴

### SUMMARY

In one aspect there is provided an antisense oligonucleotide capable of binding to exon 46 of human dystrophin pre-mRNA, wherein binding of the antisense oligonucleotide takes place entirely within the region between +89 and +149 of the pre-mRNA sequence, and wherein the antisense oligonucleotide comprises at least 26 base pairs.

In one example, the antisense oligonucleotide comprises at least 27, at least 28 bases, at least 29 bases, or at least 30 bases.

In one example, the antisense oligonucleotide consists of 30 bases.

In one example, the antisense oligonucleotide is at least 70%, at least 80%, at least 90%, at least 95%, at least 96%, at least 97%, at least 98% at least 99% complementary to a sequence of exon 46 of human dystrophin pre-mRNA falling within the region.

In one example, the antisense oligonucleotide is hybridisable to a sequence of exon 46 of human dystrophin pre-mRNA falling within the region.

In one example, the antisense oligonucleotide comprises at least 26 bases of one of the following sequences Ac89 (SEQ ID NO. 32), Ac93 (SEQ ID NO. 33), or Ac119 (SEQ ID NO. 70).

In one aspect there is provided an antisense oligonucleotide capable of binding to exon 46 of human dystrophin pre-mRNA, wherein binding of the antisense oligonucleotide takes place entirely within the region between +89 and +149 of the pre-mRNA sequence, and wherein the antisense oligonucleotide comprises at least 25 base pairs, wherein the antisense oligonucleotide comprises the sequence hAc103 (SEQ ID NO. 31).

In one aspect there is provided an antisense oligonucleotide capable of binding to exon 50 of human dystrophin pre-mRNA, wherein binding of the antisense oligonucleotide takes place entirely within the region between +5 and +98 of the pre-mRNA sequence, and wherein the antisense oligonucleotide comprises at least 26 base pairs.

In one example, the antisense oligonucleotide comprises at least 27, at least 28 bases, at least 29 bases, or at least 30 bases.

In one example, the antisense oligonucleotide consists of 30 bases.

In one example, the antisense oligonucleotide is at least 70%, at least 80%, at least 90%, at least 95%, at least 96%, at least 97%, at least 98% at least 99% complementary to a sequence of exon 50 of human dystrophin pre-mRNA falling within the region.

In one example, the antisense oligonucleotide is hybridisable to a sequence of exon 50 of human dystrophin pre-mRNA falling within the region.

In one example, the antisense oligonucleotide comprises at least 26 bases of one of the following sequences Ac5 (SEQ ID NO. 71), Ac19 (SEQ ID NO. 52), Ac63 (SEQ ID NO. 51), or Ac68 (SEQ ID NO. 72).

In one aspect there is provided an antisense cocktail containing 3 or more antisense oligonucleotides from Set no. 1, Set no. 2, or Set no. 3.

In one example, the antisense oligonucleotides from Set no. 1, Set no. 2, or Set no. 3, is at least 70%, at least 80%, at least 90%, at least 95%, at least 96%, at least 97%, at least 98% at least 99% complementary to the antisense oligonucleotides from Set no. 1, Set no. 2, or Set no. 3.

In one aspect there is provided a conjugate comprising an antisense oligonucleotide according to any of claims 1-14 and a carrier, wherein the carrier is conjugated to the antisense oligonucleotide.

In one aspect there is provided a conjugate according to claim 17, wherein the carrier is operable to transport the antisense oligonucleotide into a target cell.

In one aspect there is provided a conjugate according to claim 17 or 28, wherein the carrier is selected from a peptide, a small molecule chemical, a polymer, a nanoparticle, a lipid, a liposome or an exosome.

In one aspect there is provided a conjugate according to any of claims 27-19 wherein the carrier is a cell penetrating peptide.

In one aspect there is provided a conjugate according to any of claims 17-20 wherein the carrier is an arginine-rich cell penetrating peptide.

In one aspect there is provided a cell loaded with a conjugate of any of claims 17-21.

In one aspect there is provided a pharmaceutical composition comprising an antisense oligonucleotide according to any of claims 1-16, and/or a conjugate according to any of claims 17-22, and a pharmaceutically acceptable excipient.

In one aspect there is provided an antisense oligonucleotide of any one of claims 1 to 16, for use in the treatment of a muscular disorder in a subject.

In one aspect there is provided a conjugate of any one of claims 1 to 16, for use in the treatment of a muscular disorder in a subject.

In one example, the muscular disorder is a disorder resulting from a genetic mutation in a gene associated with muscle function.

In one example, the muscular disorder is Duchenne muscular dystrophy or Becker muscular dystrophy.

### BRIEF DESCRIPTION OF THE FIGURES

Embodiments of the present disclosure will now be described, by way of example only, with reference to the attached Figures.

FIG. 1. Associations between in-frame deletion (del.) mutations arising within the exons (ex) 45-55 region and consequent phenotypes. The functionality of different dystrophin forms can partially be explained with the proportion of two distinct phenotypes: DMD and BMD in an in-frame deletion. Exon 45, 46, 50, 51, 52, 53, and 55 are a frame-shifting one targeted by single-exon skipping therapies. The phenotype ratio in in-frame deletions that start/end at a concerned exon and are complete within the exons 45-55 region are considered associated with therapeutic outcome after exon skipping therapies, enabling the comparison of the estimated efficacy between exons 45-55 skipping and single-exon skipping strategies. A total of 897 patients carrying acceptable deletions that are determined by MLPA or equivalent methods were extracted from the Leiden DMD database. Patients having an exons 45-55 del. was not included in the group with ex45 or ex55. FDR-adjusted p values of 0.05 (*) or 0.01 (**) were considered to be statistically significant compared to ex45-55 del. (Fisher's exact test, Benjamini-Hochberg procedure). Odds ratio (OR) for BMD (odds of other in-frame del./odds of ex45-55 del.) and the 95% confidence intervals (CI) were calculated using the unconditional Maximum Likelihood Estimate. Statistically significant differences were set at *p<0.05 or **p<0.01.

FIG. 2. (A and B) In vitro screening of antisense PMOs for skipping individual DMD exons in the exons 45-55 region using RT-PCR Efficiencies of exon skipping were tested in an immortalized DMD muscle cell line with an exon 52 deletion (KM571) except exon 52 skipping for which a DMD muscle cell line with an exons 48-50 deletion (6594) was utilized. Most PMOs were tested at 5 μM. 10 μM was used for a single or combinational PMOs when less than 20% skipping efficiency was found at 5 μM. Black and gray bars indicate efficiency at skipping an exon using one and two kinds of PMOs, respectively. Data represent mean (SD) from three or four experiments in each. hAc, the human version of 25-mer mouse antisense oligos identified in our previous study.²⁰R, a rank with 30-mer AOs in an exon; r, a rank with 25-mer AOs in an exon; NA, not available; Ete, a PMO with eteplirsen sequence. § and # indicate values adapted from our previous reports using an identical method to the present study.^{22, 25} All the DNA electrophoresis images and individual skipping values used here are shown in FIG. 8.

FIG. 3. Schemes of exons 45-55 skipping using antisense PMO cocktails and the resulting truncated dystrophin structure (A) Dystrophin mRNA structures in immortalized DMD muscle cell lines (6311, 6594, and KM571) and a humanized mouse model, hDMD/Dmd-null, which has the normal human DMD gene, and the strategy of exons 45-55 skipping by cocktail PMOs. Boxes indicate exons. The shapes denote phase of triplet codons. Exon 48 can be skipped using 2 PMOs from the cocktail set 3. (B) A semi-functional dystrophin isoform found in patients with an exons 45-55 deletion or following exons 45-55 skipping treatment. In a schematic of wild-type dystrophin, binding domains that can partially be affected in the truncated dystrophin are shown: nNOS, the binding domain of neuronal nitric oxide synthase; ABD2, actin-binding domain 2; Lipid binding domain 2, a domain of binding to a phospholipid membrane bilayer. H, hinge region.

FIG. 4. Efficiencies of exons 45-55 skipping in immortalized DMD-patient derived skeletal muscle cells treated with cocktails of combinational PMOs at 1, 3, and 10 μM each tailored to their deletion mutations (A-C) DMD exons 45-55-skipping efficiencies using combinational PMOs from the cocktail set no. 3; (A) 3-exon skipping in DMD-6311 cells with ex45-52 del., (B) 8-exon skipping in 6594 cells with ex48-50 del., and (C) 10-exon skipping in KM571 cells with ex52 del. The images of tests using the PMO set nos. 1 and 2 are available in FIG. 9A-C. M, 100 bp marker; NT, non-treated; Mock, a mock 31-mer PMO at 10 μM. (D-F) Quantification of exons 45-55-skipping induced by combinational PMOs from the cocktail set nos. 1, 2, and 3; (D) 3-exon skipping against ex45-52 del., (E) 8-exon skipping against ex48-50 del., and (F) 10-exon skipping against ex52 del. Efficiency (%) of exons 45-55 skipping following treatment was normalized by that of spontaneous one observed in non-treated cells. Data represent the mean (SD) from three independent experiments. * p<0.05, ** p<0.01 compared to the next lower PMO dosage in the same cocktail set. tt p<0.01 compared to the cocktail set 1 at the same PMO dosage. p<0.05, p<0.01 compared to the cocktail set 2 at the same dosage (Tukey—Kramer test).

FIG. 5. Dystrophin restoration in DMD muscle cells treated with 3-, 8- or 10-exon skipping using cocktail PMOs. Rescued dystrophin in (A) DMD-6311 cells treated with 3 PMOs, (B) 6594 cells with 8 PMOs, and (C) KM571 cells with 11 PMOs (10 μM each) from the cocktail set no. 3 was measured by Western blotting with the anti-dystrophin C-terminal domain antibody. Total protein of 9 μg from 6311 cells and 18 μg from 6594 or KM571 cells was loaded. The band images with the cocktail set nos.

1 and 2 are available in FIG. 9D-F. To calculate the expression levels in DMD cells, healthy muscle cell lines, KM155 and 8220 were used for a standard curve in the range from 1.3% to 20% protein of that of DMD cells (averaged R²=0.97, SD 0.028, representatives are shown in FIG. 9G). Total protein amount of KM155 cells was adjusted to the same amount of DMD cells using the total protein of non-treated DMD cells. (D-F) Quantification of dystrophin induced by combinational PMOs from the cocktail set no. 1, 2 or 3 in (D) 6311 cells with ex45-52 del., (E) 6594 with ex48-50 del., and (F) KM571 with ex52 del. Expression levels of rescued dystrophin were normalized by that of spontaneous one observed in non-treated DMD cells and were calculated with a standard curve using the 8220 healthy muscle cells for the comparison. Data represent the mean (SD) from three independent experiments. **, p<0.01 compared to the set 1; p<0.01 compared to the set 2 (Tukey-Kramer test).

FIG. 6. In vivo exons 45-55 skipping using 12 PMOs of the cocktail set no. 3 by the intramuscular (i.m.) injection into tibialis anterior (TA) muscles of a humanized mouse model with the normal human DMD gene and without the entire mouse Dmd gene (hDMD/Dmd-null mouse) A cocktail of 12 PMOs at 20 and 100 μg in total (1.67 and 8.33 μg each PMO, respectively) was injected once into left and right TA muscles of mice, respectively. One week after the injection, the muscles were harvested. The efficiency (%) of exons 45-55 skipping was analyzed by RT-PCR as shown in the bottom of the image. M, 100 bp marker. (A) Representative images of in vivo exons 45-55 skipping in individual TA muscles of hDMD/Dmd-null mice. (B) Quantification of exons 45-55 skipped mRNA levels as represented by the mean (SEM). n=5 in injected TA muscles, n=4 in control TA muscles. The statistical significance was set at *p<0.05 (Dunnett's test).

FIG. 7. Genotype-phenotype associations in patients harboring large deletion mutations (≥1 exon) (A) The occurrence frequency of deletion mutations completing within DMD exons 45-55 region. Other regions define ones where deletions start or end at an exon out of the exons 45-55 region; e.g., deletions of ex42-45 and ex53-63 fall into “Others”. (B) The ratio of DMD and BMD patients with deletion mutations in the entire DMD gene (exons 1-79), ex45-55 region and other regions. Deletions starting at exon 1 or ending at exon 79 were excluded from the analysis as they are ruled out of the definition of a frameshift. (C) The ratio of out-of-frame and in-frame mutations in the region of exons 45-55. (D) Associations between frameshift mutation types and phenotypes (DMD or BMD). Out-Fr, out-of-frame; In-Fr, in-frame. (E) The reading frame rule in the regions of exons 45-55 and others. Significant differences were calculated with two-sided Fisher's exact test (2×2 contingency table).

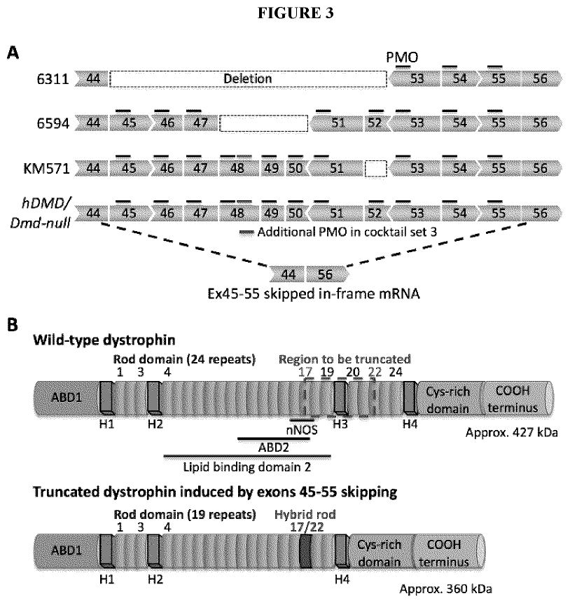

FIG. 8. Single-exon skipping efficiency of candidate PMOs for composing cocktail sets. (A-II) The efficiency of exon skipping was tested in the DMD cell line with exon 52 deletion (KM571) except exon 52 skipping for which the DMD cell line with exons 48-50 deletion (6594) was used. M, 100 bp marker, NT, non-treated. hAc, human versions of 25-mer mouse antisense oligos identified in our previous study.²⁰ The summarized result is shown in FIG. 3.

FIG. 9. Efficacy of combinational PMOs from the cocktail set 1 or 2 at skipping exons 45-55 and rescuing dystrophin expression in immortalized DMD cell lines. (A-C) Exons 45-55 skipped products induced by PMO cocktail set nos. 1 and 2, as detected in RT-PCR: (A) 3 PMOs for the DMD cells 6311 harboring ex45-52 del., (B) 8 PMOs for 6594 harboring ex48-50 del., and (C) 10 PMOs for KM571 harboring ex52 del. (D-F) Rescued dystrophin protein in the DMD cells treated with the PMO cocktail 1 or 2 as detected in Western blotting: (D) 6311, (E) 6594, and (F) KM571. Twelve μg of the total protein from DMD cells were loaded. (G) Standard curves made by the normal dystrophin protein from healthy muscle cells (KM155 and 8220) used for the calculation of rescued dystrophin levels. Representatives are shown in the range of R²=0.916−0.981 and R²=0.934−0.997 in KM155 and 8220, respectively.

FIG. 10. Western blotting in hDMD/Dmd-null mice following the intramuscular injection of the 12-PMO cocktail One week after a single intramuscular injection (i.m.) of the 12-PMO cocktail at 20 and 100 μg in total (1.67 and 8.33 μg each PMO, respectively) into tibialis anterior muscles of hDMD/Dmd-null mice, the muscles were harvested. In western blotting, the total protein of 10 μg was loaded, and the detection of the truncated dystrophin lacking the region encoded by exons 45-55 (Δex45-55) was attempted using the NSL-DYS1 antibody. Three transgenic mdx mice (Tg/mdx) were used as a positive control to detect the truncated dystrophin without the exons 45-55 region. Saline-treated muscles were used as a measure of the full-length protein.

FIG. 11. Sequences of (A) DMD exon 46, and (B) DMD exon 50. Two batches of optimization were performed, as indicated by the orange- and green-color coded PMOs. Red lines indicate antisense oligonucleotides that were designed and tested by other groups.

FIG. 12. Screening approach for exon 46, 50 skipping PMOs. Immortalized healthy (KM155) myoblasts were seeded and differentiated into myotubes. At 3 days post-differentiation, myotubes were transfected with 5 μM of an exon skipping PMO using Endoporter reagent. Total RNA was harvested from cells 5 days later, for use in RT-PCR analysis of exon skipping.

FIG. 13. Skipping efficacy of exon 46 skipping PMOs. (A-C) Exon 46 skipping PMOs were transfected into immortalized healthy (KM155) myotubes as indicated in FIG. 12. An RT-PCR gel image result showing exon 46 skipping with the second batch of exon 46-skipping PMOs is shown. Exon skipping efficiencies were quantified and plotted from both batches of PMOs (batch 1, blue; batch 2, white). Ac93 appears to have the best skipping efficacy of those tested. The bottom table lists the actual exon skipping efficiency values (ES) compared to the ES values and ranks predicted for these PMOs by our in silico exon skipping tool.

FIG. 14. Skipping efficacy of exon 50 skipping PMOs. (A-C) Exon 50 skipping PMOs were transfected into immortalized healthy (KM155) myotubes as indicated in FIG. 12. An RT-PCR gel image result showing exon 50 skipping with the second batch of exon 50-skipping PMOs is shown, in comparison with AVI-5038. Exon skipping efficiencies were quantified and plotted from both batches of PMOs (batch 1 and AVI-5038, blue; batch 2, white). Ac5 appears to have the best skipping efficacy of those tested. The bottom table lists the actual exon skipping efficiency (ES) values compared to the ES values and ranks predicted for these PMOs by our in silico exon skipping tool

FIG. 15. RT-PCR results to quantify exon 45-55 skipping efficiency with minimized cocktails. (A-H) Immortalized healthy (KM155) or patient-derived muscle cell lines (KM571 with ex52del, 6594 with ex48-50del, and 6311 with ex45-52del) were transfected with various exon 45-55 skipping PMO cocktails at 3 days post-differentation, and then harvested 2 days later for RNA extraction and RT-PCR analysis. The compositions of the various cocktails are shown in Table 7. Red arrows (upper arrows on each gel image) indicate native, unskipped bands while green arrows (lower arrows on each gel image) indicate exon 45-55 skipped bands. n=3, error: SEM. *p<0.05, **p<0.01, ***p<0.005, ****p<0.0001 one-way ANOVA, Dunnett's vs NT. (^{φ}p<0.05, ^{φφφφ}p<0.0001 one-way ANOVA, Dunnett's vs all. NT, non-treated.

### DETAILED DESCRIPTION

These DMD genotype-phenotype associations provide the rationale of a promising therapy, exon skipping using synthetic nucleic acid analogs called antisense oligonucleotides (AOs). The current approach targets a single exon and aims to transform DMD-related out-of-frame mRNAs into in-frame ones, enabling the expression of truncated dystrophin as seen in BMD. In 2016, the first exon 51-skipping AO drug with the phosphorodiamidate morpholino oligomer (PMO) chemistry, though conditional, has been approved by the US Food and Drug Association (FDA)⁵ and clinical trials with other PMO-based AOs that target exon 45 or 53 are currently ongoing.^{6, 7} As such, PMO-mediated single-exon skipping has great promise for treating DMD.

Exons 45-55 skipping using AO cocktails is expected to overcome these limitations in single-exon skipping therapies.¹³ This multi-exon skipping strategy intends to produce a consistent dystrophin form with preserved functionality as seen in exceptionally milder or asymptomatic subjects carrying an exons 45-55 deletion.^{11, 13-18} The exons 45-55-deleted dystrophin supposedly provides a favorable outcome among patients with different mutations. As demonstrated in pre-clinical studies, the strategy is achieved by excluding all the target exons from one mRNA at the same time and thus, success in treatment largely relies on the ability of respective AOs in a cocktail to skip a given exon within the region.¹⁹⁻²¹ A ready-to-use cocktail set composed of such effective AOs could serve as tailored medication to different deletions for treating DMD patients.

In this study, for the first time, we demonstrated using the Leiden DMD database, that the exons 45-55 deletion is statistically associated with the occurrence of the mild BMD phenotype. The database analysis also revealed that a variety of AO combinations, in particular, those to skip ten and eight exons, are needed in exons 45-55 skipping therapy. Accordingly, the applicability was shown to reach to more than 65% of DMD patients with out-of- and in-frame deletions. Given the need for tailored cocktail treatment, we designed three different cocktail sets composed of PMO-based AOs using an exon-skipping efficiency predictive tool we developed previously.²² Of them, the most effective cocktail set was one formulated with select PMOs which each efficiently skipped an assigned exon in in vitro screening. Derivative PMO cocktails from this set significantly skipped up to ten exons in immortalized DMD muscle cell lines, accompanied by dystrophin restoration as represented by Western blotting. In a mouse model having the normal human DMD gene, we demonstrated the feasibility of simultaneous skipping of all eleven exons from exon 45 to 55 using the PMOs in the most effective cocktail set. This work represents the first step toward clinical application of PMO-mediated exons 45-55 skipping using a mutation-tailored cocktail approach for treating DMD.

Additionally, the present invention has identified a number of AOs that may be therapeutically effective for single exon skipping therapy of exon 46 and exon 50.

Antisense Oligonucleotide

In some aspects there is described antisense oligonucleotides having a length of at least 26 bases that bind to exon 46 of human dystrophin pre-mRNA within the region of +89 to +149 which can be used to treat muscular disorders.

In some aspects there is described antisense oligonucleotides having a length of at least 25 bases that bind to exon 46 of human dystrophin pre-mRNA within the region of +89 to +149 which can be used to treat muscular disorders.

In some aspects there is described antisense oligonucleotides having a length of at least 26 bases that bind to exon 50 of human dystrophin pre-mRNA within the region of +5 to +98 which can be used to treat muscular disorders.

In some examples, ‘antisense oligonucleotides’ may be referred to as ‘AOs’ or ‘oligos’ or ‘oligomers’.

In some examples, the antisense oligonucleotide induces skipping of exon 46 of the human dystrophin gene.

In some examples, the antisense oligonucleotide increases skipping of exon 46 of the human dystrophin gene.

In some examples, the antisense oligonucleotide induces skipping of exon 50 of the human dystrophin gene.

In some examples, the antisense oligonucleotide increases skipping of exon 50 of the human dystrophin gene.

In some examples, the antisense oligonucleotide allows expression of functional human dystrophin protein.

In some example, the antisense oligonucleotide increases expression of functional human dystrophin protein.

In some examples, the antisense oligonucleotide comprises between 25 and 30 bases.

In some examples, the antisense oligonucleotide comprises at least 25 bases, at least 26 bases, at least 27 bases, at least 28 bases, suitably at least 29 bases, or at least 30 bases.

In one example the antisense oligonucleotide consists of 30 bases.

In one example, the antisense oligonucleotide is Ac89, Ac93, or Ac119.

In one example, the antisense oligonucleotide is Ac103.

In one example, the antisense oligonucleotide is Ac5, Ac19, Ac63, or Ac68.

In some examples, the antisense oligonucleotide is presented herein, for example in Tables and/or Figures.

In some examples, the antisense oligonucleotide is synthetic, and non-natural.

In some examples, the antisense oligonucleotide may be made through the well-known technique of solid phase synthesis.

In some examples, the antisense oligonucleotide is an antisense oligonucleotide analogue.

The term ‘oligonucleotide analogue’ and ‘nucleotide analogue’ may refer to any modified synthetic analogues of oligonucleotides or nucleotides respectively that are known in the art.

Examples of oligonucleotide analogues include, but are not limited to, peptide nucleic acids (PNAs), morpholino oligonucleotides, phosphorothioate oligonucleotides, phosphorodithioate oligonucleotides, alkylphosphonate oligonucleotides, acylphosphonate oligonucleotides, phosphoramidite oligonucleotides, tricyclo-DNA, and 2′methoxyethyl oligonucleogides.

In some examples the antisense oligonucleotide comprises morpholino subunits.

In some examples, the antisense oligonucleotide is a morpholino antisense oligonucleotide.

In some examples, the antisense oligonucleotide comprises morpholino subunits linked together by phosphorus-containing linkages. In a specific example, the antisense oligonucleotide is a phosphoramidate or phosphorodiamidate morpholino antisense oligonucleotide.

The terms ‘morpholino antisense oligonucleotide’ or ‘PMO’ (phosphoramidate or phosphorodiamidate morpholino oligonucleotide) refer to an antisense oligonucleotide analog composed of morpholino subunit structures, where (i) the structures are linked together by phosphorus-containing linkages, for example one to three atoms long, for example two atoms long, and for example uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide.

In some examples, the antisense oligonucleotide comprises phosphorus-containing intersubunit linkages joining a morpholino nitrogen of one subunit to a 5′ exocyclic carbon of an adjacent subunit.

In some examples, the antisense oligonucleotide comprises phosphorus-containing intersubunit linkages in accordance with the following structure (I):

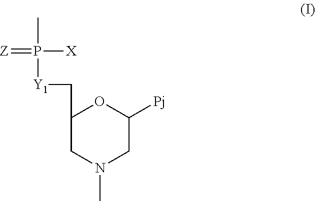

wherein:

Y1 is —O—, —S—, —NH—, or —CH2—;

Z is O or S;

Pj is a purine or pyrimidine base-pairing moiety effective to bind, by base-specific hydrogen bonding, to a base in a polynucleotide; and

X is fluoro, optionally substituted alkyl, optionally substituted alkoxy, optionally substituted thioalkoxy, amino, optionally substituted alkylamino, or optionally substituted heterocyclyl.

Optionally, variations can be made to the intersubunit linkage as long as the variations do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to the phosphorus may be unsubstituted, monosubstituted, or disubstituted with (optionally substituted) lower alkyl.

### Binding of the Antisense Oligonucleotide

In some aspects, there is described an antisense oligonucleotide capable of binding within the region +89 and +149 of exon 46 of human dystrophin pre-mRNA.

In some aspects, there is described an antisense oligonucleotide capable of binding within the region +5 and +98 of exon 50 of human dystrophin pre-mRNA.

By ‘capable of binding’ it is meant that the antisense oligonucleotide comprises a sequence with is able to bind to human dystrophin pre-mRNA in the region stated.

In some examples, the antisense oligonucleotide is complementary to a sequence of human dystrophin pre-mRNA in the region stated.

In some examples, the antisense oligonucleotide comprises a sequence which is complementary to a sequence of human dystrophin pre-mRNA in the region stated.

The antisense oligonucleotide and a sequence within the region +89 to +149 of exon 46 of human dystrophin pre-mRNA, or the antisense oligonucleotide and sequence within the region of +5 and +98 of exon 50 of human dystrophin pre-mRNA, are complementary to each other when a sufficient number of corresponding positions in each molecule are occupied by nucleotides which can hydrogen bond with each other and thereby cause exon skipping, suitably exon skipping of exon 46 or exon 50, respectively.

Accordingly, ‘hybridisable’ and ‘complementary’ are terms which are used to indicate a sufficient degree of complementarity or pairing such that stable and specific binding occurs between the antisense oligonucleotide and a sequence within region +89 to +149 of exon 46 of human dystrophin pre-mRNA or within region +5 and +98 of exon 50 of human dystrophin pre-mRNA .

In some examples, the antisense oligonucleotide is sufficiently hybridisable and/or complementary to a sequence within region +89 to +149 of exon 46 of human dystrophin pre-mRNA to induce exon skipping, suitably exon skipping of exon 46, or the antisense oligonucleotide is sufficiently hybridisable and/or complementary to a sequence within region +5 to +98 of exon 50 of human dystrophin pre-mRNA to induce exon skipping, suitably exon skipping of exon 50.

In some example, the antisense oligonucleotide may not be 100% complementary to a sequence within region of +89 to +149 of exon 46 of human dystrophin pre-mRNA or +5 to +98 of exon 50 of human dystrophin pre-mRNA . However, suitably the antisense oligonucleotide is sufficiently complementary to avoid non-specific binding.

In some examples the antisense oligonucleotide is at least 70%, at least 80%, at least 90%, at least 95%, at least 96%, at least 97%, at least 98% at least 99% complementary to a sequence within the region +89 to +149 of exon 46 of human dystrophin pre-mRNA or within region +5 and +98 of exon 50 of human dystrophin pre-mRNA.

It will be appreciated that in order for the antisense oligonucleotide to be capable of binding, it does not require that the entire length of the antisense oligonucleotide binds to the human dystrophin pre-mRNA. It will be appreciated that a portion of the antisense oligonucleotide may not bind to the human dystrophin pre-mRNA, for example the 5′ or the 3′ ends of the antisense oligonucleotide. However, in accordance with some aspects, the parts of the antisense oligonucleotide which are bound to the human dystrophin pre-mRNA must fall within the region of +89 to +149 of exon 46, or within the region of +5 to +98 if exon 50.

In some examples, the antisense oligonucleotide is hybridisable to a sequence within the region of +89 to +149 of exon 46 of human dystrophin pre-mRNA, or the region of +5 to +98 of exon 50 of human dystrophin pre-mRNA.

In some examples, the antisense oligonucleotide is sufficiently hybridisable to a sequence within the region of 0 +89 to +149 of exon 46 of human dystrophin pre-mRNA, or the region of +5 to +98 of exon 50 of human dystrophin pre-mRNA to cause exon skipping of exon 46 or exon 50, respectively.

### Human Dystrophin

In some aspects there is described to a therapeutic antisense oligonucleotide for use in the treatment of muscular disorders, particularly dystrophin disorders such as DMD.

The mRNA encoding dystrophin in muscular dystrophy patients typically contains out-of-frame mutations (e.g. deletions, insertions or splice site mutations), resulting in frameshift or early termination of the translation process, so that in most muscle fibres no functional dystrophin is produced.

In some examples, the antisense oligonucleotide(s) herein triggers exon skipping to restore the reading frame of the dystrophin mRNA. In some examples, the antisense oligonucleotide triggers exon skipping of exon 46 or 50 to restore the reading frame of the dystrophin mRNA. In some examples, restoration of the reading frame restores production of a partially functional dystrophin protein.

In some examples, the partially functional dystrophin is a truncated dystrophin protein.

In some examples, the truncated dystrophin protein is the same dystrophin protein produced in patients suffering from the less severe muscular disorder; BMD. Muscular Disorder

In one aspect there is described a use of therapeutic antisense oligonucleotides in the treatment of muscular disorders.

The muscular disorder is selected from any muscular disorder resulting from a genetic mutation.

In some examples, the muscular disorder is selected from any muscular disorder resulting from a genetic mutation in a gene associated with muscle function.

In some examples, the muscular disorder is selected from any muscular disorder resulting from a genetic mutation in the human dystrophin gene.

In some examples, the muscular disorder is selected from any muscular dystrophy disorder.

In some examples, the muscular disorder is selected from Duchenne muscular dystrophy, Becker muscular dystrophy, congenital muscular dystrophy, Distal muscular dystrophy, Emery—Dreifuss muscular dystrophy, Facioscapulohumeral muscular dystrophy, Limb-girdle muscular dystrophy, Myotonic muscular dystrophy, Oculopharyngeal Muscular dystrophy.

In some examples, the muscular disorder is Duchenne Muscular

Dystrophy (DMD) or Becker Muscular Dystrophy (BMD). Carrier and Conjugate

In one aspect there is provided a conjugate of the antisense oligonucleotide with a carrier.

The carrier may comprise any molecule operable to transport the antisense oligonucleotide into a target cell, for example, into a muscle cell.

Non limiting examples of carriers may include; peptides, small molecule chemicals, polymers, nanoparticles, lipids, liposomes, exosomes or the like.

In one example, the carrier is a peptide. The peptide may be selected from viral proteins such as VP22 (derived from herpes virus tegument protein), snake venom protein such as CyLOP-1 (derived from crotamin), cell adhesion glycoproteins such as pVEC (derived from murine vascular endothelial-cadherin protein), Penetratin (Antennapedia homeodomain), Tat (human immunodeficiency virus transactivating regulatory protein) or reverse Tat, for example.

In one example, the peptide is a cell penetrating peptide.

In one example, the peptide is an arginine-rich cell penetrating peptide.

In some examples, Ian arginine-rich peptide carriers are useful. Certain arginine based peptide carriers have been shown to be highly effective at delivery of antisense compounds into primary cells including muscle cells. Furthermore, compared to other peptides, the arginine peptide carriers when conjugated to an antisense oligonucleotide, demonstrate an enhanced ability to alter splicing of several gene transcripts.

In some examples, the carrier has the capability of inducing cell penetration of the antisense oligonucleotide within at least 30%, 40%, 50%, 60%, 70%, 80%, 90%, or 100% of cells of a given cell culture population.

In some examples, the carrier has the capability of inducing cell penetration of the antisense oligonucleotide within at least 30%, 40%, 50%, 60%, 70%, 80%, 90%, or 100% of muscle cells in a muscle cell culture.

In some examples, conjugation of the carrier to the antisense oligonucleotide may be at any position suitable for forming a covalent bond between the carrier and the antisense oligonucleotide or between the linker moiety and the antisense oligonucleotide. For example, conjugation of a carrier may be at the 3′ end of the antisense oligonucleotide. Alternatively, conjugation of a carrier to the antisense oligonucleotide may be at the 5′ end of the oligonucleotide. Alternatively, a carrier may be conjugated to the antisense oligonucleotide through any of the intersubunit linkages.

In some examples, the carrier is covalently coupled at its N-terminal or C-terminal residue to the 3′ or 5′ end of the antisense oligonucleotide.

In some examples, the carrier is coupled at its C-terminal residue to the 5′ end of the antisense oligonucleotide.

In some examples, optionally, where the antisense oligonucleotide comprises phosphorus-containing intersubunit linkages, and the carrier is a peptide, the peptide may be conjugated to the antisense oligonucleotide via a covalent bond to the phosphorous of the terminal linkage group.

In some examples, alternatively, when the carrier is a peptide, and the antisense oligonucleotide is a morpholino, the peptide may be conjugated to the nitrogen atom of the 3′ terminal morpholino group of the oligomer.

In some examples, optionally, the carrier may be conjugated to the antisense oligonucleotide via a linker moiety. Optionally, the linker moiety may comprise one or more of: an optionally substituted piperazinyl moiety, a beta alanine, glycine, proline, and/or a 6-aminohexanoic acid residue in any combination.

In some examples, alternatively, the carrier may be conjugated directly to the antisense oligonucleotide without a linker moiety.

In some examples, the conjugate may further comprise a homing moiety.

In some examples, the homing moiety is selective for a selected mammalian tissue, i.e., the same tissue being targeted by the antisense oligonucleotide. In some examples, the homing moiety is selective for muscle tissue.

In some examples, the homing moiety is a homing peptide.

In some examples, the carrier peptide and the homing peptide may be formed as a chimeric fusion protein.

In some examples, the conjugate may comprise a chimeric peptide formed from a cell penetrating peptide and a muscle-specific homing peptide.

In some examples, optionally, the conjugate may be of the form: carrier peptide-homing peptide-antisense oligonucleotide or of the form: homing peptide-carrier peptide-antisense oligonucleotide.

In some examples, the antisense oligonucleotide may be conjugated to a carrier that enhances the solubility of the antisense oligonucleotide. In some examples, the solubility in an aqueous medium. In some examples, a carrier that enhances solubility may be conjugated to the antisense oligonucleotide in addition to a carrier operable to transport the antisense oligonucleotide. In some examples, the carrier that enhances solubility and the carrier that transports the antisense oligonucleotide may be formed as a chimeric fusion protein.

Carriers that may enhance the solubility of an antisense oligonucleotide are polymers, such as polyethylene glycol, or triethylene glycol. Pharmaceutically Acceptable Excipient

In one aspect there is described a pharmaceutical composition comprising the antisense oligonucleotide of the invention or a conjugate thereof, further comprising one or more pharmaceutically acceptable excipients.

In some examples, the pharmaceutical composition is prepared in a manner known in the art, with pharmaceutically inert inorganic and/or organic excipients being used.

The term ‘pharmaceutically acceptable’ refers to molecules and compositions that are physiologically tolerable and do not typically produce an allergic or similarly untoward reaction when administered to a patient.

In some examples, the pharmaceutical composition may be formulated as a pill, tablet, coated tablet, hard gelatin capsule, soft gelatin capsule and/or suppository, solution and/or syrup, injection solution, microcapsule, implant and/or rod, and the like.

In some examples, the pharmaceutical composition may be formulated as an injection solution.

In some examples, pharmaceutically acceptable excipients for preparing pills, tablets, coated tablets and hard gelatin capsules may be selected from any of: Lactose, corn starch and/or derivatives thereof, talc, stearic acid and/or its salts, etc.

In some examples, pharmaceutically acceptable excipients for preparing soft gelatin capsules and/or suppositories may be selected from fats, waxes, semisolid and liquid polyols, natural and/or hardened oils, etc.

In some examples, pharmaceutically acceptable excipients for preparing solutions and/or syrups may be selected from water, sucrose, invert sugar, glucose, polyols, etc.

In some examples, pharmaceutically acceptable excipients for preparing injection solutions may be selected from water, saline, alcohols, glycerol, polyols, vegetable oils, etc.

In some examples, pharmaceutically acceptable excipients for preparing microcapsules, implants and/or rods may be selected from mixed polymers such as glycolic acid and lactic acid or the like.

In some examples, the pharmaceutical composition may comprise a liposome formulation.

In some examples, optionally, the pharmaceutical composition may comprise two or more different antisense oligonucleotides or conjugates thereof. Optionally, the pharmaceutical composition may further comprise one or more antisense oligonucleotides or conjugates thereof targeting different exons, suitably different exons of the human dystrophin pre-mRNA. Optionally, the one or more further antisense oligonucleotides or conjugates thereof may target exons adjacent to exon 46 or 50 of the human dystrophin pre-mRNA. Suitably, the one or more antisense oligonucleotides or conjugates thereof targeting different exons of the human dystrophin pre-mRNA are operable, together with the antisense oligonucleotide of the invention, to restore the reading frame of dystrophin mRNA.

In some examples, optionally, the pharmaceutical composition may further comprise one or more antisense oligonucleotides or conjugates thereof targeting different genes. For example, the one or more further antisense oligonucleotides or conjugates thereof may target myostatin.

In some examples, optionally, the one or more further antisense oligonucleotides may be joined together and/or joined to the antisense oligonucleotide of the first aspect.

In some examples, optionally, the antisense oligonucleotide and/or conjugate may be present in the pharmaceutical composition as a physiologically tolerated salt. Suitably, physiologically tolerated salts retain the desired biological activity of the antisense oligonucleotide and/or conjugate thereof and do not impart undesired toxicological effects. For antisense oligonucleotides, suitable examples of pharmaceutically acceptable salts include (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

In some examples, optionally, the pharmaceutical composition may comprise, in addition to at least one antisense oligonucleotide and/or conjugate, one or more different therapeutically active ingredients. The one or more therapeutically active ingredients may be selected from, for example: corticosteroids, utrophin-upregulators, TGF-beta inhibitors, and myostatin inhibitors.

In some examples, in addition to the active ingredients and excipients, a pharmaceutical composition may also comprise additives, such as fillers, extenders, disintegrants, binders, lubricants, wetting agents, stabilizing agents, emulsifiers, preservatives, sweeteners, dyes, flavorings or aromatizing agents, thickeners, diluents or buffering substances, and, in addition, solvents and/or solubilizing agents and/or agents for achieving a slow release effect, and also salts for altering the osmotic pressure, coating agents and/or antioxidants. Suitable additives may include Tris-HCI, acetate, phosphate, Tween 80, Polysorbate 80, ascorbic acid, sodium metabisulfite, Thimersol, benzyl alcohol, lactose, mannitol, or the like. Administration

In some aspects there is described a therapeutic antisense oligonucleotide and a pharmaceutical composition comprising the therapeutic antisense oligonucleotide which are for administration to a subject.

In some examples, the antisense oligonucleotide and/or pharmaceutical composition may be for topical, enteral or parenteral administration.

In some examples, the antisense oligonucleotide and/or pharmaceutical composition may be for administration orally, transdermally, intravenously, intrathecally, intramuscularly, subcutaneously, nasally, transmucosally or the like.

In some examples, the antisense oligonucleotide and/or pharmaceutical composition is for intramuscular administration.

In some examples, the antisense oligonucleotide and/or pharmaceutical composition is for intramuscular administration by injection.

An ‘effective amount’ or ‘therapeutically effective amount’ refers to an amount of the antisense oligonucleotide, administered to a subject, either as a single dose or as part of a series of doses, which is effective to produce a desired physiological response or therapeutic effect in the subject.

In some examples, the desired physiological response includes increased expression of a relatively functional or biologically active form of the dystrophin protein, suitably in muscle tissues or cells that contain a defective dystrophin protein or no dystrophin.

In some examples, the desired therapeutic effects include improvements in the symptoms or pathology of a muscular disorder, reducing the progression of symptoms or pathology of a muscular disorder, and slowing the onset of symptoms or pathology of a muscular disorder. Examples of such symptoms include fatigue, mental retardation, muscle weakness, difficulty with motor skills (e.g., running, hopping, jumping), frequent falls, and difficulty walking.

In some examples, the antisense oligonucleotide or conjugate thereof are administered at a dose in the range from about 0.0001 to about 100 mg per kilogram of body weight per day.

In some examples, the antisense oligonucleotide or conjugate thereof are administered daily, once every 2, 3, 4, 5, 6, 7, 8, 9, 10, 11, 12, 13, 14 days, once every 1, 2, 3, 4, 5, 6, 7, 8, 9, 10, 11, 12 weeks, or once every 1, 2, 3, 4, 5, 6, 7, 8, 9, 10, 11, 12 months.

In some examples, the dose and frequency of administration may be decided by a physician, as needed, to maintain the desired expression of a functional dystrophin protein.

In some examples, the antisense oligonucleotide or conjugate thereof may be administered as two, three, four, five, six or more sub-doses separately at appropriate intervals throughout the day, optionally, in unit dosage forms. Subject

In one aspect there is described a treatment of a muscular disorder by administering a therapeutically effective amount of the antisense oligonucleotide or conjugate thereof to a subject in need thereof.

In some examples, the subject has a muscular disorder, as defined above.

In some examples, the subject is mammalian. Suitably the subject is human.

In some examples, the subject may be male or female.

In some examples, the subject is male.

In some examples, the subject is any age. However, in some examples, the subject is between the ages of 1 month old to 50 years old, between the ages of 1 years old and 30 years old, between the ages of 2 years old to 27 years old, between the ages of 4 years old to 25 years old. Increased Exon Skipping and Dystrophin Expression

In one aspect there is described a therapeutic antisense oligonucleotide for use in the treatment of muscular disorder by inducing exon skipping in the human dystrophin pre-mRNA to restore functional dystrophin protein expression.

In some examples,

In some examples, a ‘functional’ dystrophin protein refers to a dystrophin protein having sufficient biological activity to reduce the progressive degradation of muscle tissue that is otherwise characteristic of muscular dystrophy when compared to the defective form of dystrophin protein that is present in subjects with a muscular disorder such as DMD.

In some examples, a functional dystrophin protein may have about 10%, 20%, 30%, 40%, 50%, 60%, 70%, 80%, 90%, or 100% of the in vitro or in vivo biological activity of wild-type dystrophin.

In some examples, a functional dystrophin protein has at least 10% to 20% of the in vitro or in vivo biological activity of wild-type dystrophin.

In some examples, the activity of dystrophin in muscle cultures in vitro can be measured according to myotube size, myofibril organization, contractile activity, and spontaneous clustering of acetylcholine receptors.

Animal models are also valuable resources for studying the pathogenesis of disease, and provide a means to test dystrophin-related activity. Two of the most widely used animal models for DMD research are the mdx mouse and the golden retriever muscular dystrophy (GRMD) dog, both of which are dystrophin negative. These and other animal models can be used to measure the functional activity of various dystrophin proteins.

In some examples, ‘exon skipping’ refers to the process by which an entire exon, or a portion thereof, is removed from a given pre-processed RNA (pre-mRNA), and is thereby excluded from being present in the mature RNA that is translated into a protein.

In some examples, the portion of the protein that is otherwise encoded by the skipped exon is not present in the expressed form of the protein.

In some examples, therefore, exon skipping creates a truncated, though still functional, form of the protein as defined above.

In some examples, the exon being skipped is an exon from the human dystrophin gene, which may contain a mutation or other alteration in its sequence that otherwise causes aberrant splicing.

In some examples, the exon being skipped is exon 46 of the dystrophin gene.

In some examples, the exon being skipped is exon 50 of the dystrophin gene.

In some examples, the antisense oligonucleotide is operable to induce exon skipping in dystrophin pre-mRNA.

In some examples, the antisense oligonucleotide is operable to induce exon skipping of exon 46 in dystrophin pre-mRNA.

In some examples, the antisense oligonucleotide is operable to induce exon skipping of exon 50 in dystrophin pre-mRNA.

In some examples, the antisense oligonucleotide is operable to increase expression of a functional form of a dystrophin protein in muscle tissue, and is operable to increase muscle function in muscle tissue.

In some examples, the antisense oligonucleotide is operable to increase muscle function by at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% compared to muscle function in subjects with a muscular disorder such as DMD that have not received the antisense oligonucleotide.

In some examples, the antisense oligonucleotide is operable to increase the percentage of muscle fibres that express a functional dystrophin protein in about at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% of muscle fibres compared to subjects with a muscular disorder such as DMD that have not received the antisense oligonucleotide.

In some examples, the antisense oligonucleotide is operable to induce expression of a functional form of a dystrophin protein to a level of at least 1, 2, 3, 4, 5, 6, 7, 8, 9, 10, 15, 20, 25, 30, 25, 40, 45, or 50% of the expression of dystrophin protein in wild type cells and/or subjects.

In some examples, the antisense oligonucleotide is operable to induce expression of a functional form of a dystrophin protein to a level of at least 1, 2, 3, 4, 5, 6, 7, 8, 9, 10, 15, or 20% of the expression of dystrophin protein in wild type cells and/or subjects.

In some examples, antisense oligonucleotide is operable to induce expression of a functional form of a dystrophin protein to a level of at least 10, 15, or 20% of the expression of dystrophin protein in wild type cells and/or subjects.

In some examples, the antisense oligonucleotide is operable to induce exon 51 skipping in the dystrophin pre-mRNA to a level of at least 10%, 20%, 30%, 40%, 50%, 60%, 70%, 80%, 90%, or 100%.

In some examples, the antisense oligonucleotide is operable to induce exon 46 skipping in the dystrophin pre-mRNA to a level of between 60% to 80%.

In some examples, the antisense oligonucleotide is operable to induce exon 50 skipping in the dystrophin pre-mRNA to a level of between 60% to 80%.

An ‘increased’ or ‘enhanced’ amount may include an increase that is 1.1, 1.2, 1.5, 2, 2.5, 3, 4, 5, 6, 7, 8, 9, 10, 15, 20, 30, 40, 50 or more times the amount produced when no antisense oligonucleotide compound (the absence of an agent) or a control compound is administered under the same circumstances.

In some examples, an ‘increased’ or ‘enhanced’ amount is a statistically significant amount.

Method of the invention is conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Abstract

Mutations in the dystrophin (DMD) gene and consequent loss of dystrophin cause Duchenne muscular dystrophy (DMD). A promising therapy for DMD, single-exon skipping using antisense phosphorodiamidate morpholino oligomers (PMOs), currently confronts major issues that an antisense drug induces the production of functionally undefined dystrophin and may not be similarly efficacious among patients with different mutations. Accordingly, the applicability of this approach is particularly suited to out-of-frame mutations. Here, using an exon-skipping efficiency predictive tool, we designed three different PMO-cocktail sets for exons 45-55 skipping aiming to produce a dystrophin form with preserved functionality as seen in milder/asymptomatic individuals with an in-frame exons 45-55 deletion. Of them, the most effective set was composed of select PMOs of which each efficiently skips an assigned exon in cell-based screening. Its combinational PMOs fitted to different deletions of immortalized DMD patient-muscle cells significantly induced exons 45-55-skipped transcripts with removing three, eight or ten exons and dystrophin restoration as represented by Western blotting. In vivo skipping of the maximum eleven human DMD exons was confirmed in humanized mice. The finding indicates that our PMO set can be used as mutation-tailored cocktails for exons 45-55 skipping and treat over 65% DMD patients carrying out-of- or in-frame deletions.

### Results

Overview of Clinical Presentation in Patients with an Exons 45-55 Deletion

We first ensured their clinical profile by summarizing literature published so far, using 52 patients of which the exons 45-55 deletion was determined by Multiplex Ligation-dependent Probe Amplification (MLPA) or a combination of multiplex PCR and Southern blotting (Table 3). For profiling, five cases of patients were newly obtained from the Canadian Neuromuscular Disease Registry (CNDR). The clinical data confirmed that those with this large deletion consistently exhibit mild to asymptomatic phenotypes and retain walking ability up to the late seventies. In all patients referred, elevated serum creatine kinase levels were present. Some patients were reported to manifest cardiac involvement but not respiratory symptoms.

### Association Between Exons 45-55 Deletion and BMD Phenotype

We analyzed the DMD genotype-phenotype associations using the registries of 4,929 patients with deletions determined by MLPA or equivalently accurate methods, and consequent phenotypes from the Leiden DMD database. The analyses revealed that more than 67% of deletion mutations occur within exons 45-55 (FIG. 7A). More BMD phenotype and in-frame-type deletions were found in this region compared to those in other regions ranging from exon 2 to 44 or from 56 to 78 (FIGS. 71B and C). In the exons 45-55 region, in-frame deletions were statistically more associated with BMD, and the reading frame rule held at a higher 97% in BMD compared to other regions (FIGS. 71D and E).

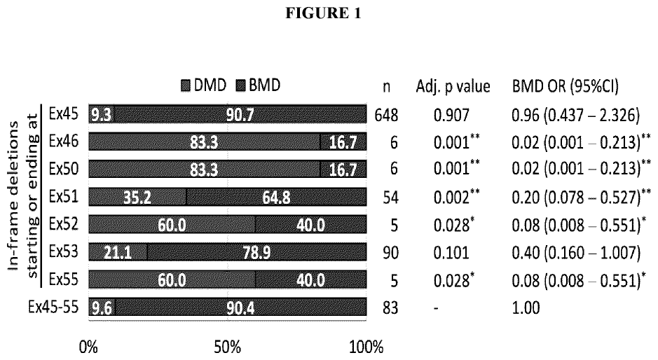

Phenotypes found in patients with in-frame deletions involving a frame-shifting exon as the first or last one in the region partially explain therapeutic outcome from a single-exon skipping.11, 17, 23 An analysis of the proportion of BMD/DMD in in-frame deletions within the region first statistically revealed that an in-frame exons 45-55 deletion is more associated with the onset of BMD compared to in-frame deletion types starting or ending at an exon 46, 50, 51, 52 or 55 (FIG. 1). In the group of deletions that start or end at exon 45 or 53, no statistical difference was found (proportions in individual deletions are available in). In exon 55-related in-frame deletions, the exons 45-55 deletion involved more than 90% patients as being BMD (75 out of 83), while in other deletions ending at exon 55, 3 out of 5 patients were diagnosed with DMD. The result emphasizes the therapeutic relevance of exons 45-55 removal.

Applicability of Exons 45-55 Skipping Thrapy uUing cCmbinational AO cCcktails

Table 1 represents the applicability of AO cocktails for exons 45-55 skipping therapy to DMD deletion types and phenotypes from the Leiden DMD database. It was revealed that this approach can be applied to ˜65% of all patients having deletions (n=4,929). Approx. 69% and 45% of DMD patients carrying out-of- and in-frame deletions, respectively, are amenable to exons 45-55 skipping. In DMD with out-of-frame deletions, cocktails of 10 AOs in combination permit treatment of the largest population (18% of cases), followed by that of 8 AOs (11%). In DMD with in-frame deletions, cocktail 7 AOs were the most required (9%). In terms of the phenotypes, ˜65% and —70% of DMD and BMD patients having deletions are treatable with exons 45-55 skipping.

Design of Cocktail Sets with PMO-Modified AOs

To establish a therapeutic set of AOs that can be used as mutation-tailored cocktails, we designed and compared three different cocktail sets composed of PMO-based AOs, each of which contained PMOs assigned to an exon in the exons 45-55 region (Table 2). Individual PMOs composing these sets were optimized through a screening method using in silico and in vitro approaches, i.e., predicted and actual exon skipping efficiencies, respectively. Cocktail set no. 1 consisted of 11 30-mer PMOs that were selected to prevent dimerization between PMOs which may affect the therapeutic activity and safety in use. Set no. 2 consisted of 11 25-mer PMOs that are mostly the human analog versions of sequences used in our previous studies involving mouse vivo-PMOs that showed efficient exons 45-55 skipping of the mouse Dmd gene.^{20, 24} Cocktail set no. 3 is composed of 12 30-mer PMOs, including 2 PMOs for exon 48 skipping, of which each was found to be the most effective for skipping an assigned AO in cell-based screening using RT-PCR. The screening process is described in the following sections:

In silico screening of AO sequences: First, we designed 151 to 413 AO sequences against each exon in the exon 45-55 region, covering all possible target sites in individual exons. According to our AO screening model,^{22, 25} AO length was determined with 30- and 25-mer for PMO modification. Exon skipping efficiencies of all sequences were predicted using robust algorithms we have previously developed,²² providing us with a final ranking that can be used for the selection of AO sequences. In all exons tested, predicted skipping efficiencies of 30-mer AOs were higher than those of 25-mer AOs.

We also calculated the dimerization potential between AO sequences using a formula for the Gibbs free energy of binding (dG). The dimer formation relates to lowered exon skipping efficiency and an increase in potential side effects.²⁶⁻²⁸ Along with AO ranking, the composition of set no. 1 was determined with 30-mer PMOs having potentially less chances of dimerization, as represented by a higher integration value of dG −363 kcal/mole than that of −504 kcal/mole in set no. 3. Using the NCBI BLAST, the theoretical specificity of selected AO sequences to a target DMD exon was confirmed by the absence of mRNA sequences of other genes identical to the entire AO sequences in the results; 100% identity was found with less than 56% and 84% of the query covering for 30- and 25-mer sequences, respectively. Sequence searching with the GGGenome server revealed fewer genome sites similar to AO sequences with an increase in the length (Table 4), indicating that longer 30-mer AOs can work in a more sequence-specific manner and have less potential for affecting untargeted transcripts including non-coding RNAs that mostly exist in nuclei where AOs work.

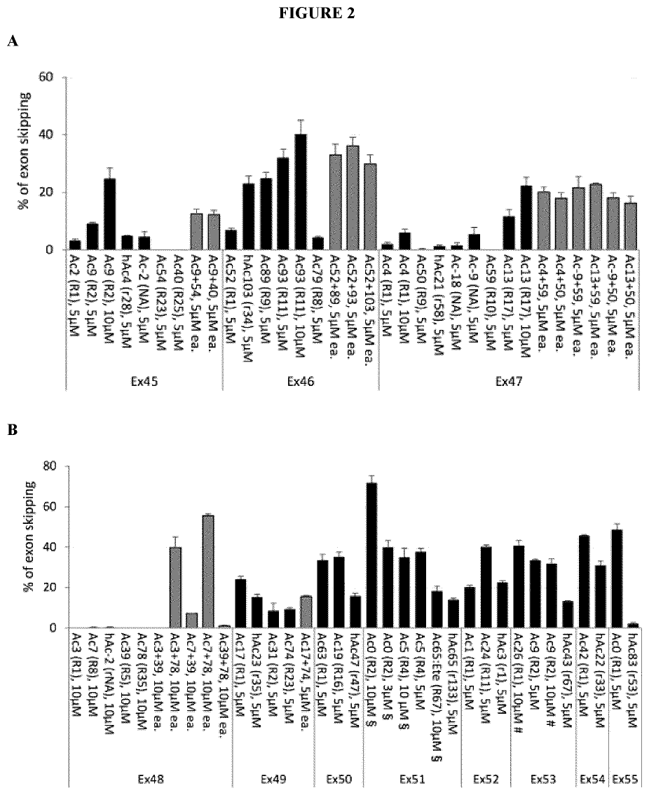

In vitro screening of PMO-based AOs: We next evaluated the actual exon skipping efficiencies of AO sequences selected through in silico screening. All the AOs tested here were prepared as PMOs that are a promising chemistry as to effectiveness and safety in patients.^{5, 7} In in vitro screening, a DMD patient-derived immortalized skeletal muscle cell line carrying an exon 52 deletion (ID: KM571) was used for testing single-exon skipping except exon 52 skipping, for which that with an exons 48-50 deletion (ID: 6594) was used. PMO-mediated single-exon skipping as represented by RT-PCR was efficiently induced in all the target exons (FIGS. 2 and 8). PMOs that resulted in greater than 20% exon skipping efficiency when tested at 5 or 10 μM were selected to compose cocktail set no. 3, according to our previous studies, i.e., in vitro PMO activity can increase up to 10 μM and such skipping levels can be considered associated with dystrophin production as detected by Western blotting.^{22, 25} Effective 30-mer PMOs in each exon were found within the top 17 in the ranking of exon skipping efficiencies. While efficient exon skipping was found using a single PMO in most exons, exon 48 skipping was remarkably induced with 2 different PMOs. Thus, for cocktail set no. 3, we included 2 PMOs for skipping exon 48. Such a synergistic effect was also observed for the skipping of exons 46 and 47. PMOs with 25-mer that were previously optimized with vivo-PMOs²⁰ were not as effective to induce exon skipping efficiencies over 20%, except one for exon 46 skipping and one for exon 52 skipping that was first in the ranking.

### Exons 45-55 Skipping by Tailored PMO Cocktail Approach in DMD Muscle Cells

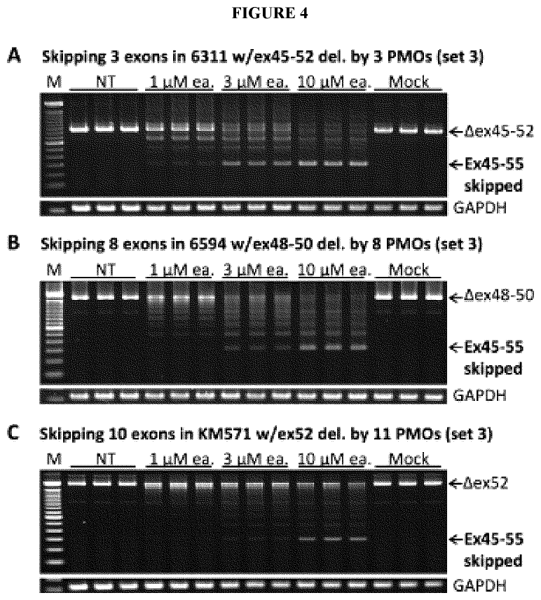

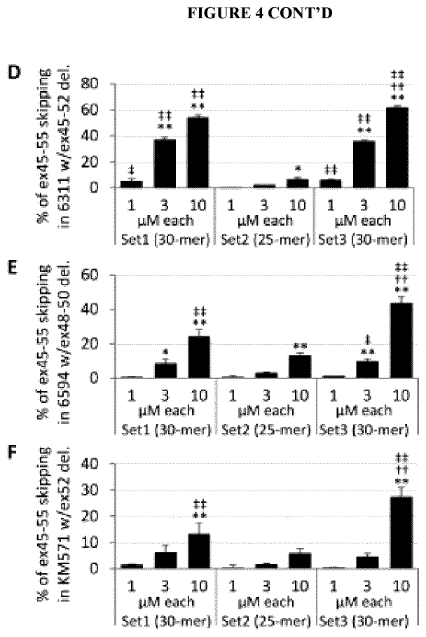

To assess the therapeutic potential of cocktail set nos. 1, 2, and 3 in exons 45-55 skipping, we tested its derivative combinational PMO cocktails tailored to treat the different DMD deletions of exon(s) 45-52, 48-50, and 52 in immortalized DMD muscle cell lines referred to as 6311, 6594, and KM571, respectively (FIG. 3). In RT-PCR analyses, as represented by the expression of exons 45-55-skipped transcripts, all the derivative cocktails prepared from set no. 1, 2 or 3 induced 3-, 8-, and 10-exon skipping at doses of 1, 3, and 10 μM per PMO (FIGS. 9A-C for the set nos. 1 and 2; FIG. 4A-C for the set no. 3). In all the cocktail sets/combinations, the efficiency of exons 45-55 skipping was increased in a dose-dependent manner. PMO cocktail set no. 3 was significantly effective at skipping multiple exons in DMD cells, compared to the other two sets (FIG. 4D-F); using the cocktails at 10 μM each, levels of exons 45-55-skipped mRNA reached up to 61%, 43% and 27% on average in 3-, 8-, and 10-exon skipping applications, respectively. In the course of testing all the cocktail sets and combinations used, various intermediate transcripts that included in-frame and out-of-frame species were produced. The expression patterns of these intermediates, however, were unchanged between different concentrations, indicating that the activity of respective PMOs in a cocktail still proportionately increases depending on the dose.

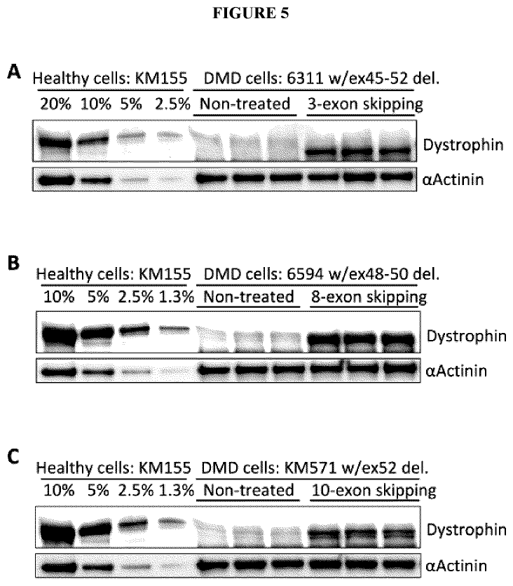

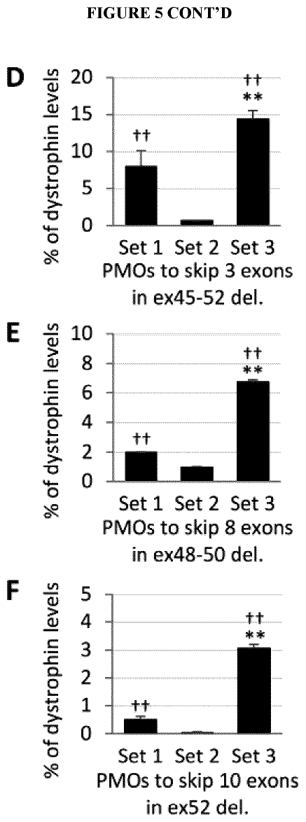

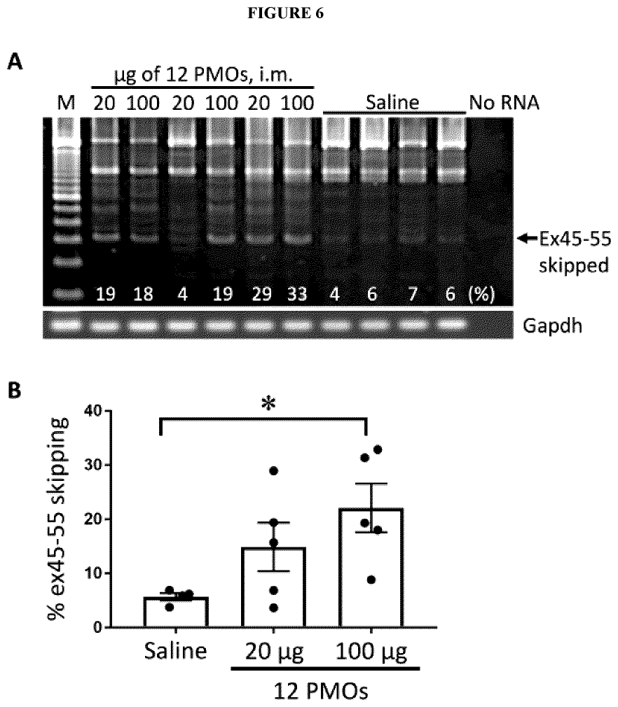

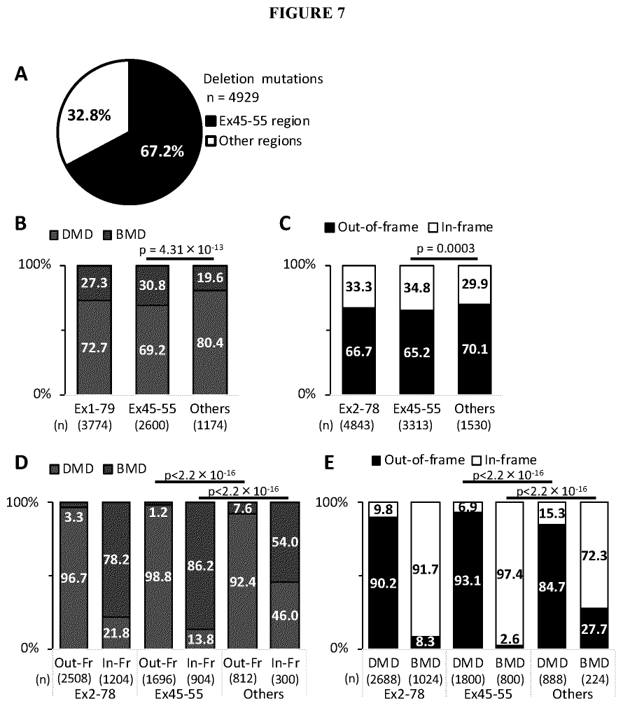

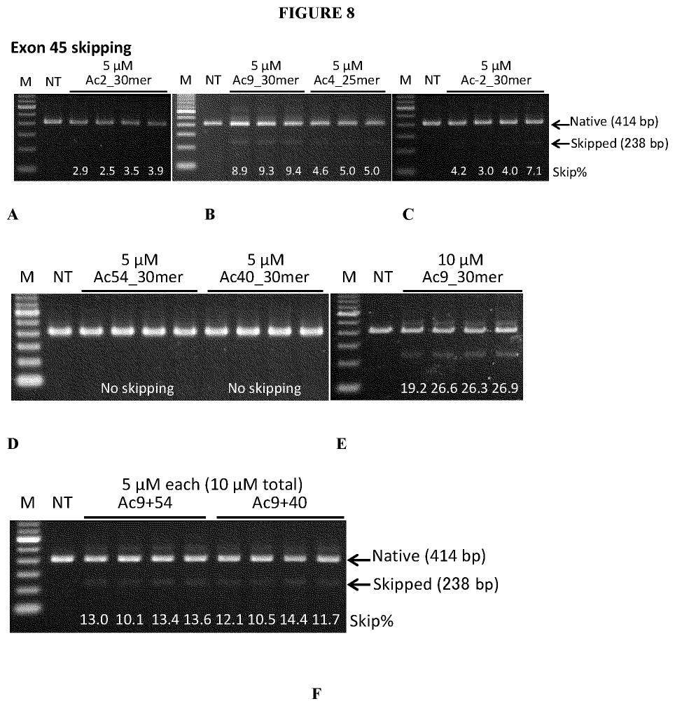

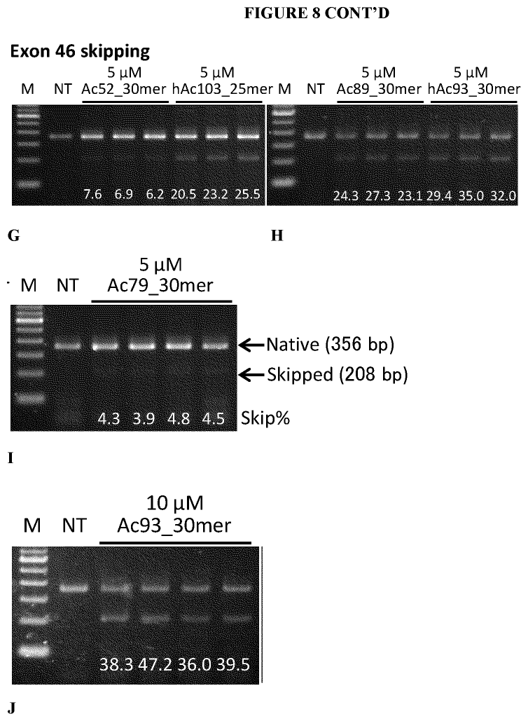

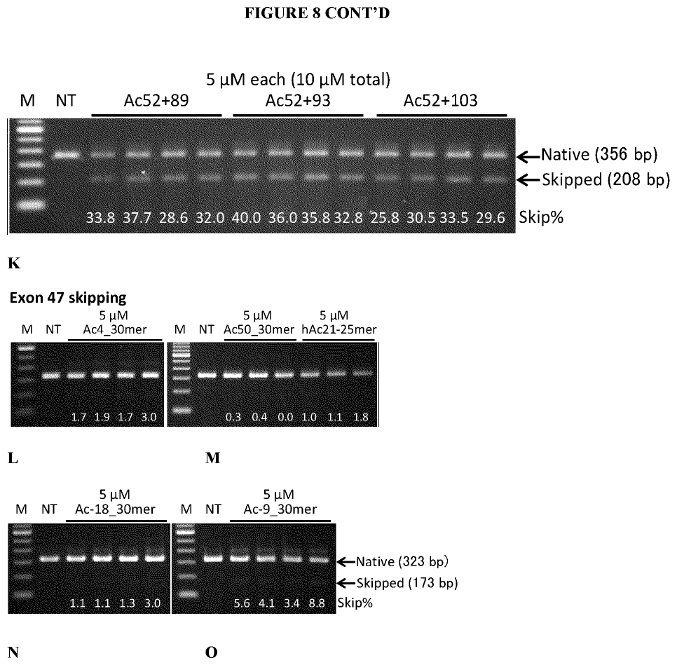

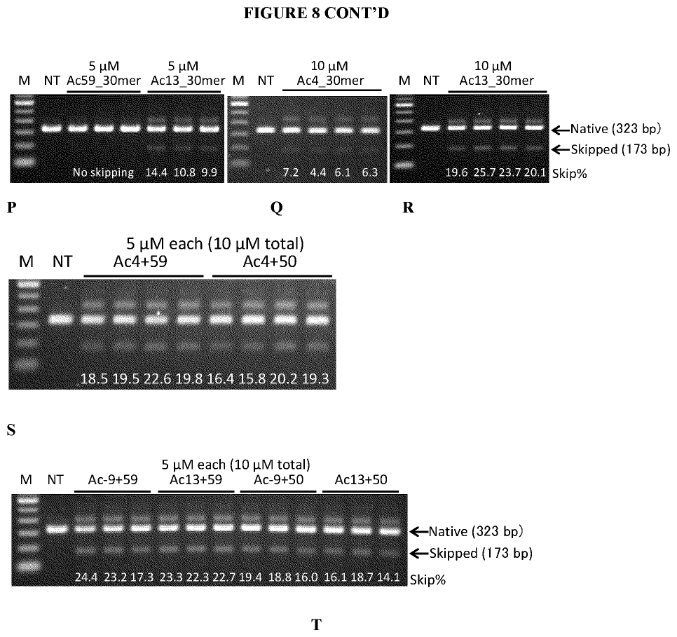

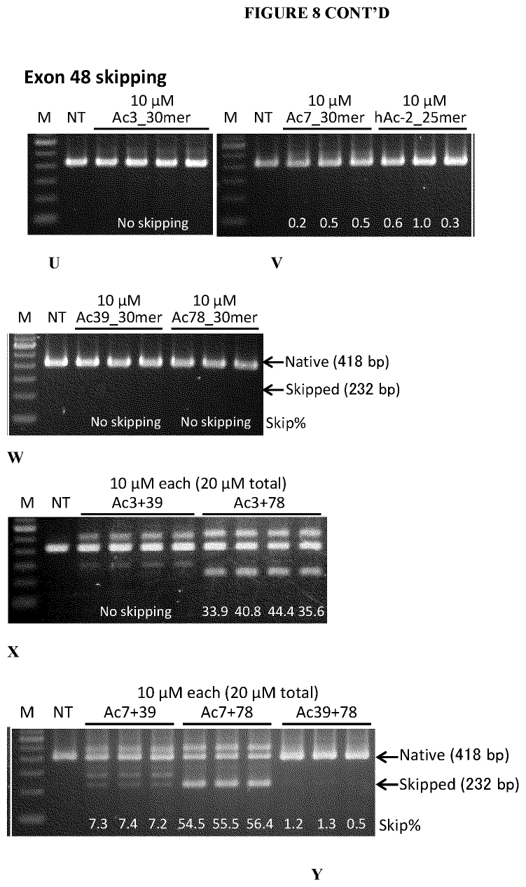

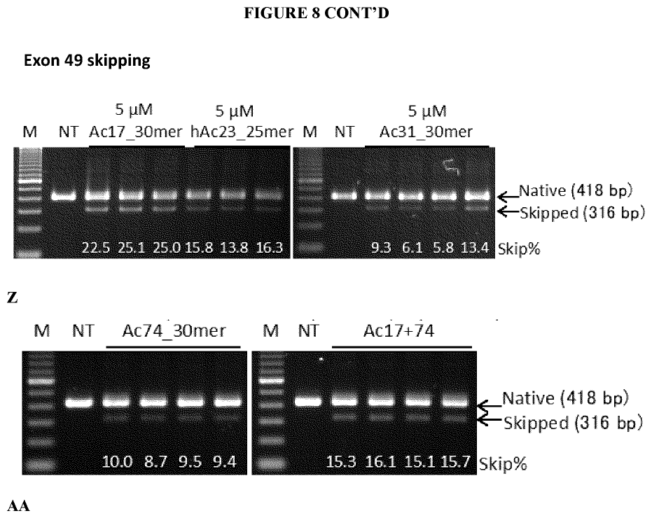

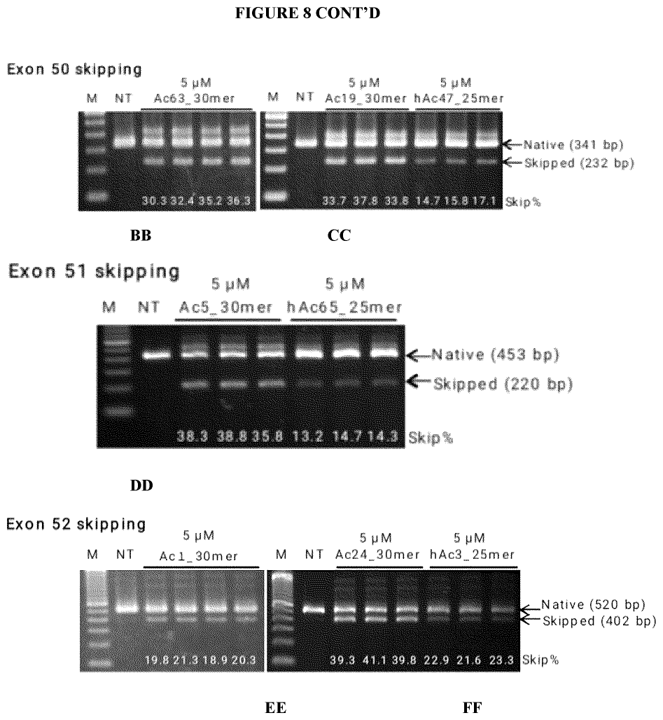

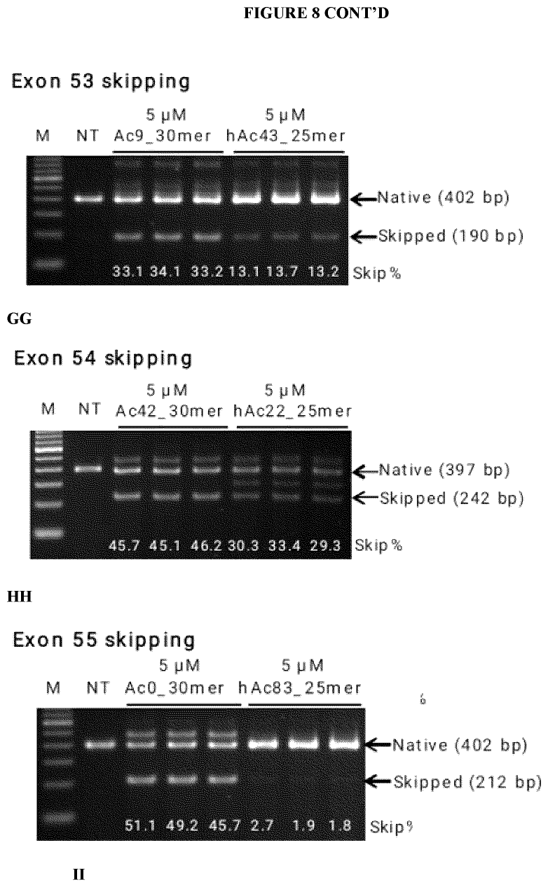

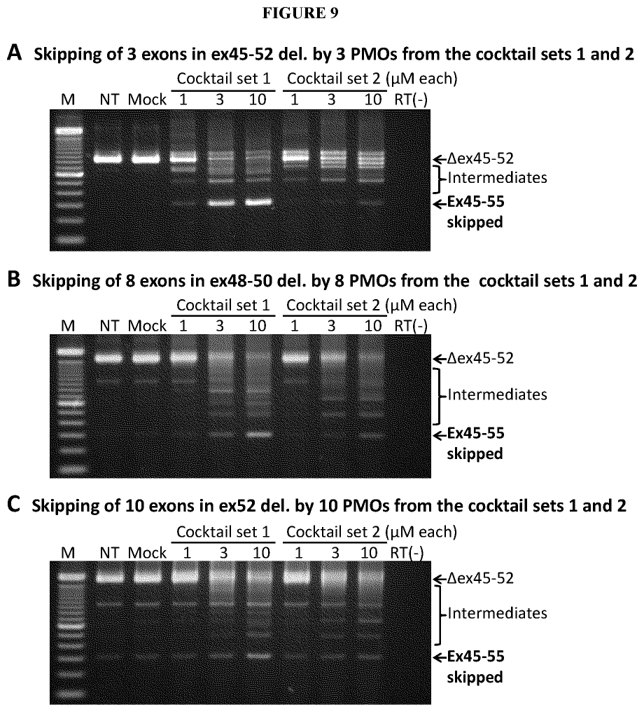

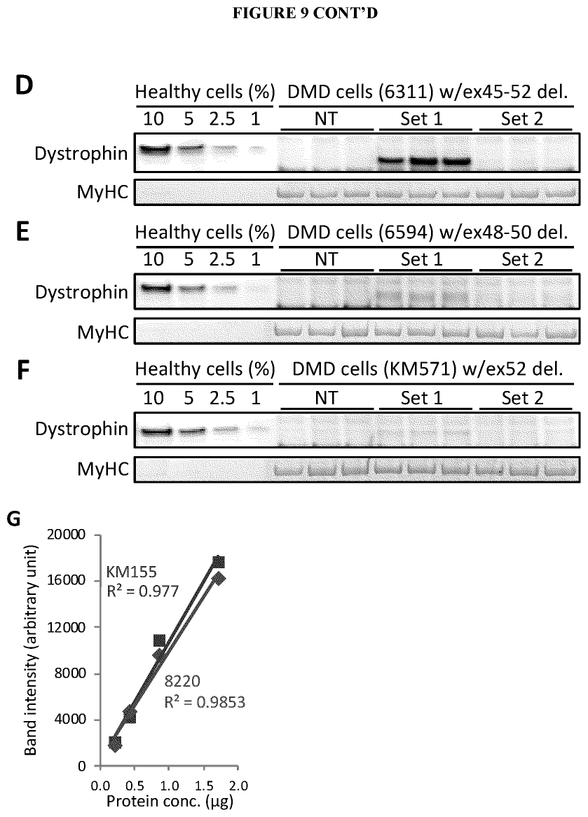

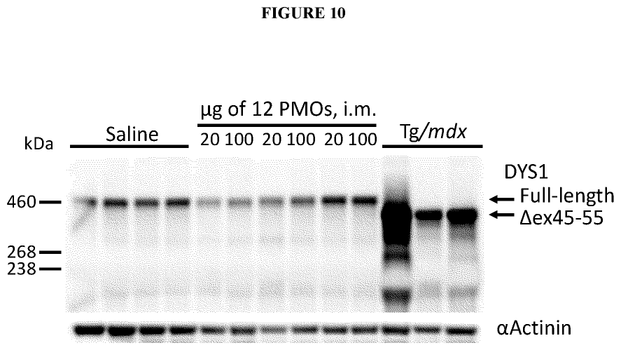

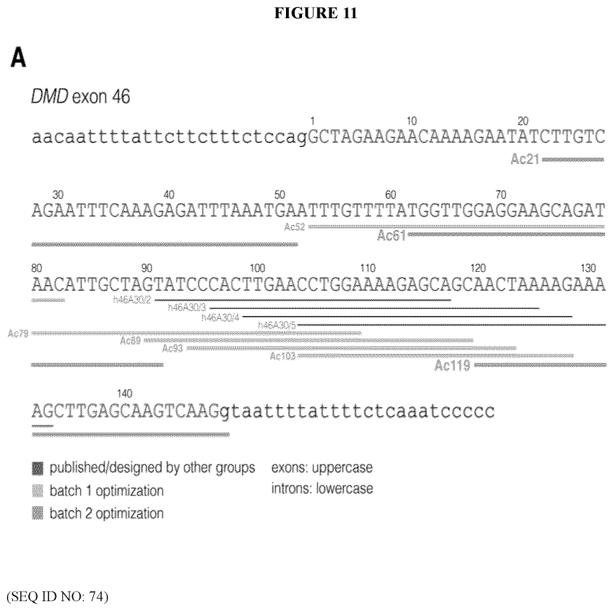

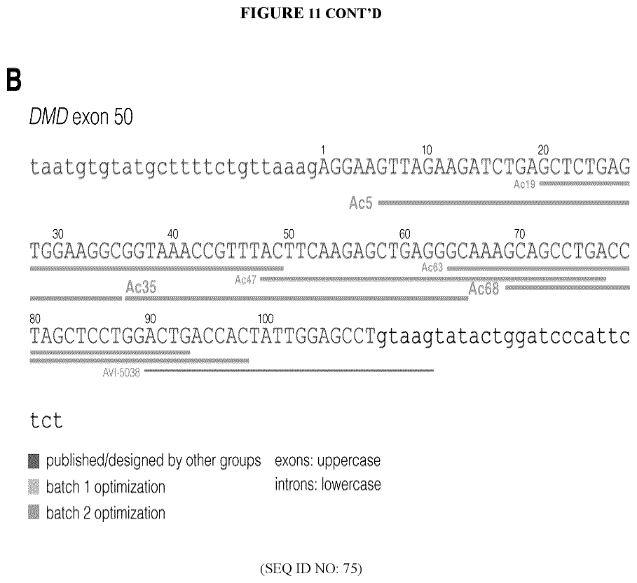

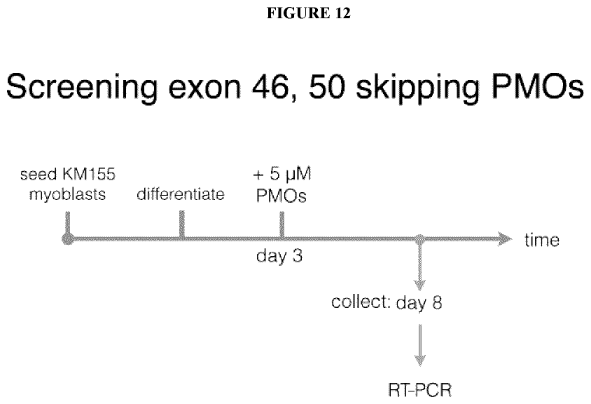

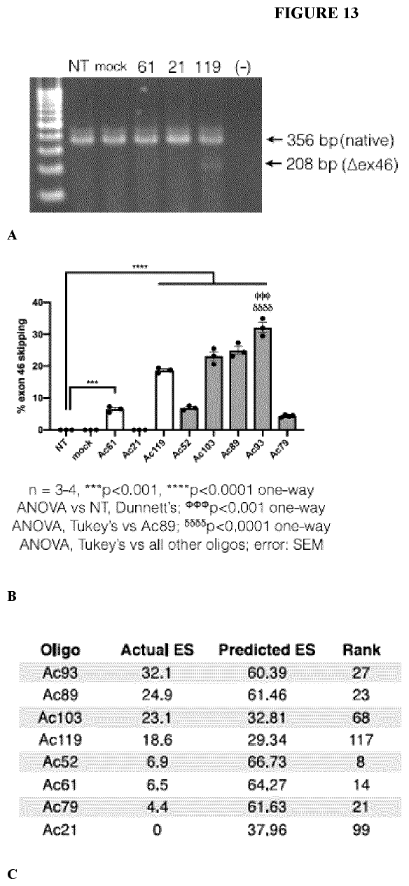

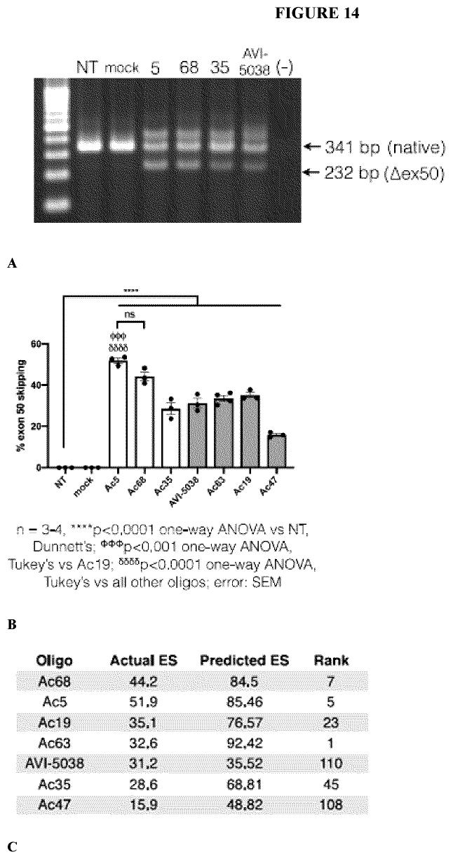

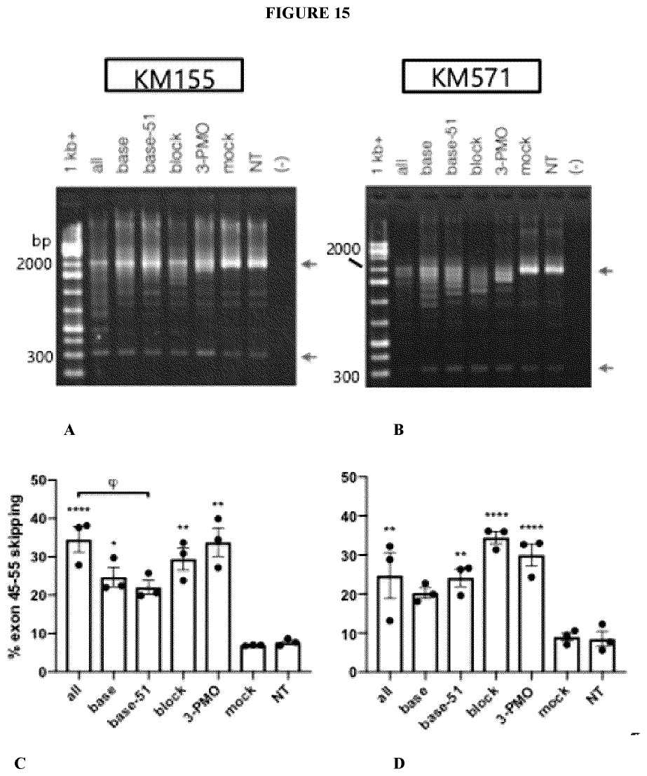

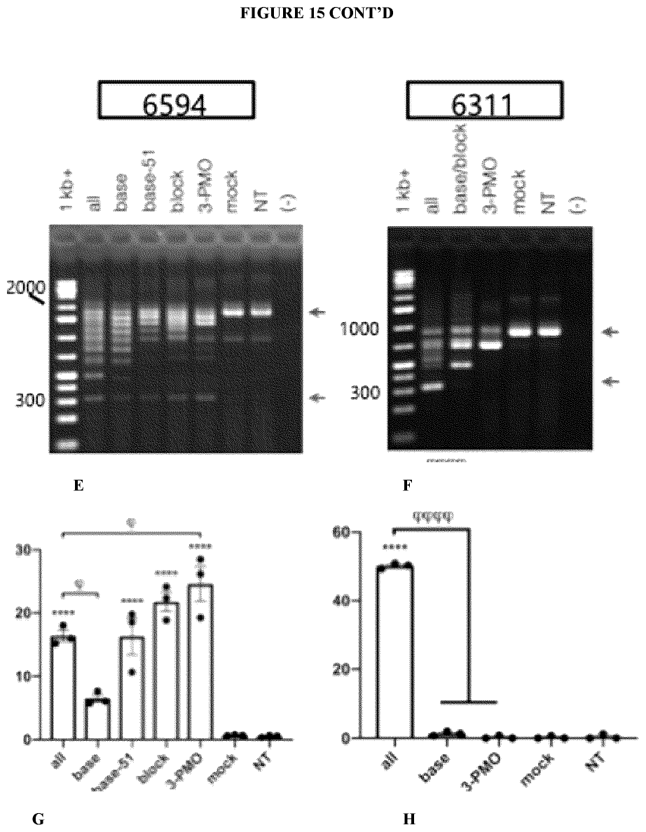

Consistent with the RT-PCR result, dystrophin restoration was induced in DMD muscle cells treated with derivative PMO cocktails prepared from set no. 3 when tested at a dose of 10 μM per PMO (FIG. 5A-C). In the treatment of DMD cells with set no.3 PMO cocktails for 3-, 8-, and 10-exon skipping, 14%, 7% and 3% dystrophin of normal levels were induced, respectively (FIG. 5D-F). For set no. 1 (FIG. 9D-F), appreciable dystrophin bands were found only in 6311 cells treated with the 3-PMO cocktail, while 8- and 10-exon skipping using this set produced very small amounts of dystrophin in 6594 and KM571 cells, having less than 2% of normal levels. Using set no. 2, no substantial dystrophin bands were detected in any of the three DMD cells. Compared to set nos. 1 and 2, the significant effect of set no. 3 on skipping 3, 8, or 10 exons was confirmed.

In Vivo Efficacy of the Cocktail PMOs to Skip 11-human DMD Exons in a Mouse Model

Finally, we tested the in vivo efficacy of exons 45-55 skipping using PMO set no. 3 in a humanized mouse model called the hDMD/Dmd-null mouse that has the normal human DMD gene and lacks the entire mouse Dmd gene.²⁵ In this model, to induce exons 45-55-skipped transcripts, all eleven exons need to be simultaneously skipped from the DMD mRNA, which allows for evaluating the maximum capability of set no. 3 in in vivo exons 45-55 skipping. In this test, we intramuscularly injected 12 PMOs composing set no. 3 as a cocktail at the dose of 20 or 100 μg in total (1.67 and 8.33 μg of each PMO) into tibialis anterior muscles. One week after injection, muscles were harvested for analyses of exon skipping using RT-PCR and of truncated dystrophin production by Western blotting. The result showed exons 45-55 skipping efficiency of 15% and 22% on average at the low and high dose, respectively (FIG. 6). Although skipping levels were variable between PMO-treated samples, the dose-dependent effect of the 12-PMO cocktail on skipping exons 45-55 in vivo was confirmed. Consistent with a previous report,²⁹ spontaneous DMD exons 45-55-skipped transcripts were detected in saline-treated control muscles. In Western blotting, the dystrophin of the treated hDMD/Dmd-null mice was detected only at the expected molecular size of the full-length protein as confirmed using samples from saline-treated mice and transgenic mice expressing the truncated dystrophin protein lacking the exons 45-55 region 30 (FIG. 10).

### Discussion

As shown through analyses of clinical overview and genotype-phenotype association (Table 3 and FIG. 1), skipping of the entire exons 45-55 region possesses strong rationale to be applied for DMD therapy. An important finding from the analysis is that the in-frame deletion of the entire exon 45-55 region is statistically associated with the milder BMD when compared to other in-frame deletions arising within the region. Given this clinical relevance of the exons 45-55 deletion, here, we have successfully developed the complete set of PMO-based AOs for exons 45-55 skipping from which the PMOs can be used in combination tailored to different DMD mutations. One key feature of our cocktail set is a use of the PMO chemistry that has been deemed sufficiently safe for human use.' Accordingly, the present study outlined a screening model for success in developing multi-exon skipping PMOs. Our model involves a series of in silico pre-screening allowing for the rational selection of PMO sequences, which uses the prediction analyses of exon skipping efficiency and potential off-target effects (Table 4), followed by an in vitro screening with immortalized DMD muscle cells that determines PMOs to be included in a cocktail set (FIG. 2; primers in Table 6). With the substantial activity of individual PMOs to skip a given exon, the feasibility of the tailored cocktail approach has been proved by the successful skipping of 3, 8, and 10 exons (FIG. 4), accompanied with dystrophin rescue (FIG. 5), in three different DMD muscle cells having acceptable mutations. Importantly, while PMO-based AOs are typically incompetent for in vivo application, in particular, multi-exon skipping,³¹⁻³³ our cocktail PMOs achieved in a humanized mouse model the removal of the maximum 11 exons from the normal human DMD mRNA (FIG. 6).

The present results revealed that the effect of cocktail PMOs is largely dependent on the sequence/target RNA position of each, highlighting the need for a rigorous selection of respective PMOs to compose a cocktail set as done here. For the selection process, a reliable in silico pre-screening is indispensable to reasonably narrow down the options of AO sequences moving on to a subsequent cell-based screening, out of a few hundred candidates designed as encompassing an entire exon region . Here, this pre-screening allowed for the selection of highly effective PMOs against all the exons in the exons 45-55 region, except exon 48, using the ranking of predicted exon skipping efficiencies with our in silico tool, ^{22, 25} as validated by the actual efficiencies in DMD cells (FIG. 2). Although useful to find effective PMOs for individual exons in the region of interest, the current tool has some issues including that the use is limited to 30- and 25-mer PMO sequences and that the synergistic effect of AOs on the removal of an exon, as found in exon 48 skipping, cannot be predicted. With the improvement of the predictive algorithms, in silico pre-screening will increase the opportunity to discover more effective PMOs not only for exons 45-55 skipping but also for different multi-exon skipping strategies.² Such advanced algorithms are also expected to enable the optimization of AO sequences used with other AO chemistries that have greater bioavailability in multi-exon skipping, e.g., peptide-conjugated PMOs.³⁴

Along with the optimal design of PMO sequences, appropriate patient cell models in the subsequent in vitro screening are an essential tool to evaluate and develop multi-exon skipping PMOs. Because rescued dystrophin levels are a primary biomarker of therapeutic benefits from exon skipping therapies, cell models need to allow for the quantification of the protein by Western blotting that is suggested by the FDA in clinical trials with eteplirsen.³⁵ We have previously shown in DMD patient fibroblast-converted myotubes, the induction of exons 45-55 skipped transcripts using 5- and 6-exon skipping PMO cocktails,²¹ but this transdifferentiated cell model was not enough to quantify the efficiency at exons 45-55 skipping and dystrophin rescue due to low differentiation ability of the cells. In contrast, immortalized DMD muscle cells enabled the quantification of dystrophin restoration by Western blotting in the test with exons 45-55 skipping PMOs. Because such DMD muscle cell lines available are currently limited, the development of those with different mutations amenable to exons 45-55 skipping are required to further confirm the application of tailored approaches with a cocktail set.

Following cell-based screening, the in vivo efficacy of the selected AOs needs to be examined in an appropriate animal model, such as the humanized mouse model used in this study. Our hDMD/Dmd-null mouse model has the advantage of allowing for the assessment of the activity of human-specific AOs in vivo without being confounded by expression of homologous mRNA derived from the mouse Dmd gene. In this model, however, treatment effects such as dystrophin rescue, histological amelioration, and functional recovery cannot be examined because of the lack of dystrophic pathology. The hDMD/Dmd-null mouse model also holds normal muscle membrane permeability that can be associated with the lowered efficiency of AO uptake. Another concern is that the reactivity of the normal DMD transcript to AOs may be different from the mutated versions found in patients. These conditions may affect the estimation of the effectiveness of human AOs in patient muscles. Indeed, the dose-dependent effect of the PMO cocktail was unclear in the healthy mouse model (FIG. 6). As a possible solution to these limitations, dystrophic hDMD mouse models having a mutation in the human DMD gene have been developed by crossing with mdx mice that have a nonsense mutation in the mouse Dmd gene. However, murine dystrophin transcripts are still present in these mice, which may pose difficulties in skipping evaluation as described previously.^{36, 37} To assess the potential benefit of AOs designed for patients, and in particular, dystrophin rescue levels, the development of dystrophic humanized mouse models, in which mutations in the human DMD gene cause dystrophic phenotypes and the mouse Dmd gene is absent, will be required.

With the database analysis, we revealed that cocktail AOs for skipping 10 exons are the most required combination to treat DMD deletions, accounted for approx. 17% of those (Table 1). In this study, we have demonstrated the 10-exon skipping in a DMD muscle cell line with the fourth most common single deletion, an exon 52 deletion (FIGS. 4 and 5).² Based on the definite effect of individual PMOs in the cocktail set no. 3 on skipping an assigned exon (FIG. 2), the PMO set has the potential for being adapted to other 10-exon skipping approaches targeting different single exon deletions, in particular, an exon 45 deletion that creates the largest population of DMD (approx. 6%). This possibility can be further supported by the 11-exon skipping in vivo shown in a mouse model with the human DMD gene (FIG. 6). As such, an exons 45-55-skipping cocktail set is versatile in that it can treat more than 65% of DMD patients with deletions (Table 1), whether they are single (e.g. Δ45, Δ51, Δ52) or multiple (e.g. Δ45-50, Δ45-52, Δ48-50) exon deletions, and whether they are out-of-frame or in-frame. In this study, theoretical applicability of this approach to BMD with deletions was also shown, opening a potential avenue of treatment for 70% of the cases, in particular, those with severe phenotypes and cardiac impairment that is a leading cause of death.³⁸

While exons 45-55 skipping is expected to lead to similar therapeutic outcomes among patients regardless of mutation patterns within amenable boundaries, a concern regarding the truncated dystrophin produced from exon skipping is the potential structural change it may create in the binding site of neuronal nitric oxide synthase (nNOS) encoded by exons 42-45 (FIG. 3B). nNOS and its metabolite NO play a crucial function in directing numerous physiological activities of muscle, such as contractile force and blood flow regulation.³⁹ In BMD patients, reduced expression of nNOS and its mislocalization from the sarcolemma to the cytoplasm have been identified.^{16, 40} A recent study with a transgenic mdx mouse model that carries the human DMD gene with a deletion of the exons 45-55 region demonstrated normalized activity of nNOS in muscles expressing truncated dystrophin as seen following exons 45-55 skipping therapy despite nNOS remaining mislocalized in the cytoplasm.³⁹ In this humanized mouse, muscle histology and function were also comparable to wild-type mice. The observed rescue effect with the truncated dystrophin may be partially associated with the amino acid sequence similarity of the hybrid rod domain 17/22 encoded by exons 44/56 to the native rod domain 17 by exon 45 in the nNOS binding site.⁴¹ In addition, the binding sites of F-actin and the sarcolemmal lipid layer are partially affected by the exons 45-55 deletion,^{42, 43} which suggests that the resulting dystrophin can alter sarcolemmal stability. A hybrid rod similar to the native rod domain 17 composed of three α-helices has been computationally predicted in some in-frame deletions such as the deletions of exons 45-48, 45-51, and 45-55.⁴⁴ Of them, the exons 45-55 deleted dystrophin has a structural resemblance to the native protein with 16 rod domains, from the hinge 2 to the next hinge 4 (FIG. 3B). A future challenge will be to address how the truncation of dystrophin impacts interactions with its binding partners and, consequently, on muscle function. This will help in better understanding the possible effects of exons 45-55 skipping as a therapy.

An issue in PMO cocktail approaches is that the efficiency of exons 45-55 skipping is lowered with an increase in the number of target exons or AOs in a cocktail. In the test using both cocktail set nos. 1 and 3 comprising 30-mer PMOs, 3- and 10-exon skipping induced the highest and lowest efficiencies, respectively (FIG. 4). This event did not occur with the 25-mer PMO set, probably due to low activity in exons 45-55 skipping. To skip the entirety of exons 45 to 55, all AOs in a cocktail have to simultaneously bind their target exons of the same pre-mRNA but such will not always be the case. In the current cocktail approach using one-to-one interaction of an AO with an exon of a target, the unequable binding of multiple AOs to a pre-mRNA is unavoidable, decreasing the efficacy of the intended multi-exon skipping. Although the dose escalation of PMOs can improve the chance of simultaneous binding of different PMOs, this also increases that of off-target effects in vivo. A possible solution to this issue may be to remove the exons 45-55 region as one or a few exon blocks from the pre-mRNA. Encouragingly, endogenous exons 45-55 skipped mRNAs have been identified in the normal DMD gene.²⁹ By revealing a mechanism for this spontaneous multi-exon skipping phenomena, exon-block skipping using minimal PMOs can become a practical approach in exons 45-55 skipping therapy. The strategy will also reduce a concern associated with the formation of unintended intermediately skipped transcripts, as found after multi-exon skipping (FIGS. 4 and 6) that may have unexpected impacts on therapeutic efficacy.

Finally, drug development regulation is another challenge to surmount for the clinical translation of tailored cocktail approaches with exons 45-55 skipping AOs. Currently, there is no specific regulatory guidance for the development of cocktail drugs using multiple AOs targeting different RNA positions in a gene. In this context, an FDA guidance, Codevelopment of Two or More New Investigational Drugs for Use in Combination, has been issued on June 2013,⁴⁵ which may partially provide some leads for the cocktail AO drug development. Referring to this guidance, it is desired to demonstrate that the greater efficacy and better toxicity profile of exons 45-55 skipping AO cocktails to single-exon skipping AOs in an in vivo (preferable) or in vitro model with mutations amenable to both strategies. Second, if the exons 45-55 skipping AOs in the cocktail set were to be adapted for patients with different mutation types, clinical trials would need to be respectively performed to separate cocktail compositions, i.e., to the number of mutation patterns, which can count 62 of the combination cocktails for 36 out-of-frame and 26 in-frame deletion patterns found in the region. However, it is in practice difficult to design such clinical trials with sufficient subjects. One significant issue is that some cocktail compositions induce harmful out-of-frame transcripts in healthy volunteers. One solution to these is to simply use the complete exons 45-55 skipping cocktail as a single agent regardless of mutation type in the region. However, compared to such a cocktail that inevitably contains non-therapeutic AOs targeting exons deleted in the patient, it is evident that tailored cocktail approaches using only AOs targeting exons that patients retain have a lower risk of side effects.

In this study, we conclude, inter alia, that PMO-mediated exons 45-55 skipping is doable in tailored cocktail approaches and has a potential for treating patients with DMD arising from out-of- and in-frame deletion mutations. The approach, however, still needs to overcome certain challenges. These include, among others, determining the functional superiority of exons 45-55 skipped dystrophin, and the efficacy and safety profile in in vivo models such as transgenic animals with dystrophic pathology arising from human DMD mutations, as well as dealing with current drug development regulations.^{2, 45} It is also to be noted that patients with other mutation types, e.g., duplication and point mutations, require this methodology as some of those can be corrected only by skipping multiple exons.^{46, 47} With more research on the approach, we expect that mutation-tailored AO cocktails will bec ome a treatment modality not only for DMD but also other genetic disorders such as dysferlinopathy with which patients can receive more therapeutic benefit from the functional correction of a causative protein.⁴⁸

### Materials and Methods

### Ethics Statement

Experiments using human cells and animals in this study were performed with approval from the Ethics Committee for the Animal Care and Use Committee (ACUC) of the University of Alberta and National Center of Neurology and Psychiatry (NCNP). Clinical data of patients enrolled in the Canadian Neuromuscular Disease Registry (CNDR) were reviewed with the approval of the Health Research Ethics Board of the University of Alberta (Pro00059937). Patients

Five new Canadian cases with DMD exons 45-55 deletion were obtained from the CNDR for this study. The information of the new cases: date at an examination, ambulatory ability, and cardiac involvement, were summarized together with that of cases previously published (Table 3).

### Genotype-Phenotype Associations and Applicability of Cocktail Treatment

A total of 16,032 patients in the Leiden Open Variation Database (LOVD v.3.0, https://databases.lovd.nl/shared/genes/DMD) were reviewed (accessed Jun. 22, 2018). Of all these patients, 4,929 cases with large exonic deletions (≥1 exon) determined with accurate and sensitive diagnostic methods were extracted for analyses. These methods include: Multiplex Ligation-dependent Probe Amplification (MLPA), Multiplex Amplifiable Probe Hybridization (MAPH), array Comparative Genomic Hybridization (array CGH), Next Generation Sequencing (NGS), or a combination of multiplex PCR and Southern blotting. In frame type-based analyses, a total of 4,843 cases were used: 3,232 and 1,611 with out-of- and in-frame deletions, respectively; 86 cases with deletions starting and/or ending at exon 1 and/or 79, which are not applicable to the definition of a frameshift, were excluded from the analyses (FIG. 7). In phenotype-based analyses, a total of 3,712 data were analyzed: 2,688 of DMD and 1,024 of BMD. Registrations without a diagnosis of DMD or BMD were omitted from the analyses. Applicability of combinational AO cocktails was analyzed with these populations (Table 1).

### Design of Antisense Sequences

All possible AO sequences 30- or 25-mer in length were designed for each of the eleven exons within exons 45-55 . Exon skipping efficiencies of the designed sequences were quantitatively predicted using the computational tool we developed previously.²²

### Dimerization Potential of AO Sequences

The lowest free energy (dG) of binding of between AOs or individual AOs was predicted with RNAstructure web servers (version 6.0.1) (https://rna.urmc.rochester.edu/RNAstructureWeb/). Dimerization potential of AO pairs was formulated as follows: dG of an AO pair−(dG of an AO+dG of the other AO). Integrated values of dimerization dG were represented as the potential risk of using an AO cocktail.

### Specificity of AO Sequences

The specificity of AO sequences was analyzed with both plus and minus strands of the human genome (reference ID: GRCh38/hg38) in GGGenome (http://gggenome.dbcls.jp/en/hg38/); the parameter was set to explore genomic sequences that differ in 5 or 4 nucleotides with mismatches/gaps from given 30- or 25-mer AOs, respectively, which considered >16.7% difference from a given AO sequence that may lead to unexpected, off-target effects.⁴⁹

### Antisense Morpholinos and PMO Cocktails

All AO sequences experimentally tested in this study were synthesized with the PMO chemistry by Gene Tools. PMO cocktails were prepared just before use in experiments; respective PMO stock vials at 1 mM were heated at 65° C. for 10 min in order to dissociate aggregations and only PMOs required to induce exons 45-55 skipping were mixed in transfection media or saline.

### Immortalized Patient-Derived Skeletal Muscle Cells

Human-derived skeletal muscle cell lines were obtained with the help of Dr. Francesco Muntoni of the MRC Centre for Neuromuscular Diseases Biobank (NHS Research Ethics Committee reference 06/Q0406/33, HTA license number 12198) in the context of Myobank, affiliated with Eurobiobank (European certification). Healthy and DMD patient-derived skeletal muscle cell lines were immortalized with CDK4 and Telomerase-expressing pBABE retroviral vectors as described previously.⁵⁰ The immortalized DMD muscle cell lines tested were 6311, 6594, and KM571 which have deletions of DMD ex45-52, ex48-50, and ex52, respectively. The immortalized healthy muscle cell lines KM155 and 8220 were used as controls.

### Transfection of Individual and Cocktail PMOs

Immortalized healthy and DMD skeletal muscle cells were grown and differentiated as described previously.²⁵ Briefly, cells were seeded at 1.7×10⁴/cm² in collagen type 1-coated culture plates, then cultured in a growth medium (GM): DMEM/F12 with skeletal muscle supplement mix (Promocell), 20% fetal bovine serum (Gibco), and antibiotics (50 U penicillin and 50 mg/ml streptomycin). At 80-90% confluence, media were replaced with a differentiation medium (DM): DMEM/F12 supplemented with 2% horse serum (GE Healthcare), lx insulin-transferrin-sodium selenite (ITS) solution (Sigma-Aldrich), and antibiotics. After 3 days in DM, myotube-differentiated DMD cells were transfected with a single PMO or multiple PMOs as a cocktail at 1, 3, 5, or 10 μM, each containing 6 μM Endo-porter transfection reagent (Gene Tools). The same amount of transfection reagent was used regardless of PMO amount according to the company's suggestion. Cocktails of combinational PMOs were prepared just before the transfection following the heating procedure described previously. Following the incubation with PMOs for 2 days, PMO-containing DM was replaced with regular DM. Three days later, cells were harvested for subsequent experiments.

### Humanized Transgenic Mice

Male transgenic hDMD mice with the full-length normal human DMD gene on mouse chromosome 5 (Jackson Laboratory)⁵¹ were cross-bred with female Dmd-null mice that lack the entire mouse gene in the X-chromosome.⁵² The resulting male offspring, called hDMD/Dmd-null mice (hDMD^{+/-}; Dmd-null^{-/Y}), accordingly expresses full-length dystrophin protein derived from the human DMD gene but not from the mouse Dmd gene, which imposes a limitation in assessing exon skipping treatment efficacy. The hDMD/Dmd-null mice were used at the age of 6-16 weeks for testing the in vivo efficacy of a 12-PMO cocktail at skipping 11 exons from exons 45 to 55. A humanized mdx mouse model that has an exons 45-55 deletion in the DMD gene and expresses exons 45-55 deleted human dystrophin was used as a positive control in Western blotting analysis with the muscle samples of hDMD/Dmd-null mice.³⁰

### PMO Cocktail Injections

PMO cocktails with total doses of 20 or 100 μg (1.67 or 8.33 μg per PMO, respectively) in 36 μL of saline were injected into the tibialis anterior (TA) muscles of hDMD/Dmd-null mice under anesthesia with sodium pentobarbital (Kyoritsu Seiyaku). The same amount of saline was intramuscularly injected into the TA muscles as a negative control. One week after the injection, mice were euthanized by cervical dislocation, and then the TA muscles injected were collected. Muscle samples were snap-frozen as described previously,²⁴ and stored at −80° C. until use.

### RT-PCR

Total RNA from cells and frozen TA muscle sections was extracted with Trizol reagent (Invitrogen) as described previously.²⁵ RT-PCR was performed in a 25-μL mixture containing 200 ng RNA and 0.2 μM of each primer with the SuperScript III One-Step RT-PCR System (Invitrogen), following manufacturer's instructions. Primer sequences are listed in Table 5. The cycling conditions were optimized depending on the amplicon size of native DMD mRNA in each DMD cell line, and it is as follows: 50° C. for 5-15 min; 94° C. for 2 min; 35-40 cycles at 94° C. for 15 sec, 60° C. for 30 sec, and 68° C. for 33-118 sec; and 68° C. for 5 min. GAPDH or Gapdh mRNA was detected as an internal control. PCR products were separated on a 1.5% agarose gel and visualized by SYBR Safe DNA Gel Stain (Invitrogen). Skipping percentage was calculated as

$$\frac{Skipped transcript}{Native + Skipped transcript} \times 100 for single exon skipping or \\ \frac{Exons 45 - 55 skipped transcript}{Native + Intermediates + Skipped transcripts} \times 100 for multiple exon skipping using ImageJ ( NIH ) .$$

Bands with the expected size of the transcript were excised and purified with a gel extraction kit (Promega). Sequencing reactions were performed with Big Dye Terminator v3.1 (Applied Biosystems).

### Western Blotting

Total protein from cells was extracted with RIPA buffer (Pierce Biotechnology) containing protease inhibitors (complete mini EDTA-free, Roche), and concentrations were measured by BCA assay (Pierce Biotechnology). Total protein from frozen muscle sections was prepared as previously described.24 Total protein extracts were loaded onto wells of a NuPAGE Novex 3-8% Tris-Acetate Midi Gel (Invitrogen) and separated by SDS-PAGE at 150 V for 75 min for cell samples and 150 min for tissues samples. Proteins were transferred onto a PVDF membrane (Millipore) by semidry blotting at 20 V for 70 min. The membrane was blocked with PBS containing 0.05% Tween 20 and 2% ECL advance blocking reagent (GE Healthcare) overnight at 4° C. The membrane was incubated with anti-dystrophin C-terminal domain antibody (1:2500, ab15277; Abcam) or NCL-DYS1 (1:200, Leica Biosystems) for 1 hour at room temperature. The primary antibody was detected with HRP-conjugated IgG H+L secondary antibody (1:10000, Invitrogen). Blots were visualized by electrochemiluminescence (GE Healthcare). Expression levels of the dystrophin protein induced by PMO cocktails were calculated using a calibration curve from 0.12 to 1.8 μg protein of immortalized healthy skeletal muscle cell lines, KM155 or 8220 (FIG. 9H). As a loading control and differentiation marker, α-actinin was detected using a primary antibody (Sigma-Aldrich). Myosin heavy chain (MyHC) on the post-transferred gel was stained by Coomassie Brilliant Blue as a loading control and as another indicator of muscle cell differentiation.

### Statistical Analysis

For association analyses between genotypes and phenotypes shown in FIG. 7, two-tailed Fisher's exact test (2×2 contingency table) was used with a p-value<0.05 considered to be statistically significant. Differences in phenotype proportions between exons 45-55 deletion and other in-frame deletions that start and end at exon(s) within the exons 45-55 region (FIG. 1) were computed using a two-tailed Fisher's exact test, and then the resulting p values were adjusted for multiple comparisons using the Benjamini-Hochberg procedure: false discovery rates (FDRs) of 0.05 or 0.01 were considered as a significant difference. Odds ratios (odds of BMD with other in-frame deletions/odds of that with exons 45-55 deletion) and 95% confidence intervals were calculated to quantify differences in the association between BMD and in-frame deletion mutations. Statistical tests for efficiency at skipping exons and rescuing dystrophin expression were performed using the Tukey-Kramer's or Dunnett's test. All statistical analyses were conducted with R (version 3.5.1).

TABLE 1
Applicability of exons 45-55 skipping
to patients with deletion mutations.

| | % applicability of exons 45-55 skipping to: | | | | |
|---|---|---|---|---|---|
| | | DMD | | | |
| Exon no. | | Out-of-frame | In-frame | | |
| to be | Del. total | del. | del. | DMD total | BMD |
| skipped | (n = 4929) | (n = 2425) | (n = 263) | (n = 2744) | (n = 1030) |
| 10 | 14.1 | 18.4 | 5.7 | 16.8 | 5.3 |
| 9 | 6.9 | 8.0 | 8.4 | 7.9 | 4.8 |
| 8 | 13.8 | 10.9 | 7.2 | 10.3 | 26.8 |
| 7 | 8.7 | 4.3 | 9.1 | 4.7 | 19.8 |
| 6 | 6.2 | 7.1 | 4.9 | 6.7 | 5.1 |
| 5 | 6.0 | 9.1 | 2.3 | 8.3 | 0.5 |
| 4 | 2.3 | 2.5 | 2.3 | 2.4 | 1.4 |
| 3 | 3.7 | 6.1 | 0.4 | 5.4 | 0.1 |
| 2 | 1.8 | 0.0 | 4.2 | 0.4 | 6.5 |
| 1 | 1.8 | 2.6 | 0.0 | 2.3 | 0.1 |
| Total | 65.2 | 69.1 | 44.5 | 65.3 | 70.4 |

Deletion (del.) total includes patients diagnosed with DMD or BMD, and those not determined with either. Deletion types in DMD consist of deletions in the region from exon 2 to 78 where the reading frame rule is applied. DMD total and BMD include patients carrying deletions in exons 1-79.

TABLE 2
PMO sequences composing cocktail sets and its rank with exon skipping
efficiency predicted in a computational tool

| | | | Rank | Predicted | SEQ |
|---|---|---|---|---|---|
| | | | within | skipping | ID |
| Cocktail Name | | AO sequence (5′ to 3′) | an exon | % | NO: |
| Set no. 1 | Ex45_Ac9_30mer | GACAACAGTTTGCCGCTGCCCAATGCCATC | 2 | 76.2 | 25 |
| | Ex46_Ac52_30mer | GTTATCTGCTTCCTCCAACCATAAAACAAA | 1 | 66.7 | 30 |
| | Ex47_Ac50_30mer | GCACTTACAAGCACGGGTCCTCCAGTTTCA | 9 | 53.0 | 36 |
| | Ex48_Ac7_30mer | CAATTTCTCCTTGTTTCTCAGGTAAAGCTC | 8 | 65.0 | 43 |
| | Ex49_Ac17_30mer | ATCTCTTCCACATCCGGTTGTTTAGCTTGA | 1 | 90.0 | 47 |
| | Ex50_Ac19_30mer | GTAAACGGTTTACCGCCTTCCACTCAGAGC | 20 | 76.6 | 76 |
| | Ex51_Ac5_30mer | AGGTTGTGTCACCAGAGTAACAGTCTGAGT | 4 | 73.0 | 55 |
| | Ex52_Ac24_30mer | GGTAATGAGTTCTTCCAACTGGGGACGCCT | 25 | 90.1 | 77 |
| | Ex53_Ac9_30mer | GTTCTTGTACTTCATCCCACTGATTCTGAA | 2 | 73.9 | 62 |
| | Ex54_Ac42_30mer | GAGAAGTTTCAGGGCCAAGTCATTTGCCAC | 1 | 62.0 | 64 |
| | Ex55_Ac0_30mer | TCTTCCAAAGCAGCCTCTCGCTCACTCACC | 1 | 120.4 | 66 |
| Set no. 2 | hEx45_Ac4_25mer | TGCCGCTGCCCAATGCCATCCTGGA | 4 | 42.7 | 26 |
| | hEx46_Ac103_25mer | CTTTTAGTTGCTGCTCTTTTCCAGG | 34 | 32.8 | 31 |
| | hEx47_Ac21_25mer | ATTGTTTGAGAATTCCCTGGCGCAG | 58 | 8.2 | 37 |
| | hEx48_Ac-2_25mer | TTCTCAGGTAAAGCTCTGGAAACCT | NA | NA | 44 |
| | hEx49_Ac23_25mer | AATCTCTTCCACATCCGGTTGTTTA | 31 | 41.9 | 48 |
| | hEx50_Ac47_25mer | CTGCTTTGCCCTCAGCTCTTGAAGT | 44 | 36.4 | 53 |
| | hEx51_Ac65_25mer | ACATCAAGGAAGATGGCATTTCTAG | 133 | −5.4 | 57 |
| | hEx52_Ac3_25mer | GCCTCTGTTCCAAATCCTGCATTGT | 1 | 74.6 | 60 |
| | hEx53_Ac43_25mer | ATTCAACTGTTGCCTCCGGTTCTGA | 67 | 7.3 | 63 |
| | hEx54_Ac22_25mer | GCCACATCTACATTTGTCTGCCACT | 33 | 12.8 | 65 |
| | hEx55_Ac83_25mer | GCAGTTGTTTCAGCTTCTGTAAGCC | 53 | 32.7 | 67 |
| Set no. 3 | Ex45_Ac9_30mer | The same as the AO in the set 1 | 2 | 76.2 | |
| | Ex46_Ac93_30mer | AGTTGCTGCTCTTTTCCAGGTTCAAGTGGG | 11 | 60.4 | 33 |
| | Ex47_Ac13_30mer | GTTTGAGAATTCCCTGGCGCAGGGGCAACT | 17 | 49.2 | 41 |
| | Ex48_Ac7_30mer | The same as the AO in the set 1 | 8 | 65.0 | |
| | Ex48_Ac78_30mer | CAGATGATTTAACTGCTCTTCAAGGTCTTC | 35 | 44.5 | 46 |
| | Ex49_Ac17_30mer | The same as the AO in the set 1 | 1 | 90.0 | |
| | Ex50_Ac19_30mer | The same as the AO in the set 1 | 16 | 76.6 | |
| | Ex51_Ac0_30mer | GTGTCACCAGAGTAACAGTCTGAGTAGGAG | 2 | 80.1 | 54 |
| | Ex52_Ac24_30mer | The same as the AO in the set 1 | 11 | 90.1 | |
| | Ex53_Ac26_30mer | CCTCCGGTTCTGAAGGTGTTCTTGTACTTC | 1 | 75.2 | 61 |
| | Ex54_Ac42_30mer | The same as the AO in the set 1 | 1 | 62.0 | |
| | Ex55_Ac0_30mer | The same as the AO in the set 1 | 1 | 120.4 | |

TABLE 3
Clinical presentations of BMD patients with the exons 45-55 deletion

| | | Years | | | Cardiac | Respiratory | CK | |
|---|---|---|---|---|---|---|---|---|
| No. | Test | at exam | Severity ^{a} | Ambulant | involvement | involvement | (IU/L) | Ref |
| 1 | MLPA | 2 | Asymptomatic | Yes | No | na | 600-3500 | 11 |
| 2 | MLPA | 5 | Oligosymptomatic | Yes | na | na | 20145 | 14 |
| 3 | MLPA | 7 | Presymptomatic | Yes | No | No | Elevated | 18 |
| 4 | MLPA | 7 | Asymptomatic | Yes | No | No | Elevated | 13 |
| 5 | MLPA | 8 | Presymptomatic | Yes | No | No | Elevated | 18 |
| 6 | MLPA | 9 | Asymptomatic | Yes | No | No | Elevated | 13 |
| 7 | Del/dup test | 11 | na | Yes | No | na | na | CNDR |
| 8 | MLPA | 13 | Exercise | Yes | No | No | Elevated | 13 |
| | | | intolerance | | | | | |
| 9 | MLPA | 14 | Asymptomatic | Yes | No | na | 5300 | 11 |
| 10 | MLPA | 14 | Mild | Yes | No | No | Elevated | 13 |
| 11 | MLPA | 14 | Myalgia | Yes | na | No | Elevated | 13 |
| 12 | MLPA | 17 | Presymptomatic | Yes | No | No | Elevated | 18 |
| 13 | Del/dup test | 18 | na | Yes | No | na | na | CNDR |
| 14 | MLPA | 18 | Mild | Yes | No | No | Elevated | 13 |
| 15 | MLPA | 19 | Presymptomatic | Yes | No | No | Elevated | 18 |
| 16 | MLPA | 19 | Asymptomatic | Yes | na | na | 849 | 14 |
| 17 | MLPA | 19 | Mild | Yes | No | No | Elevated | 13 |
| 18 | MLPA | 10 s-30 s | Mild | Yes | Yes | No | na | 12 |
| | | (n = 4) | | (4/4) | (1/4) | (0/4) | | |
| 19 | MLPA | 21 | Asymptomatic | Yes | na | na | 978 | 14 |
| 20 | MLPA | 23 | Mild | Yes | No | na | 2800-10000 | 11 |
| 21 | MLPA | 23 | Mild | Yes | na | na | Elevated | 16 |
| 22 | MLPA | 26 | Mild | Yes | No | na | 1000-4000 | 11 |
| 23 | MLPA | 26 | Mild | Yes | Yes | No | na | 17 |
| 24 | MLPA | 29 | Mild | Yes | na | na | Elevated | 16 |
| 25 | MLPA | 34 | Mild | Yes | na | na | Elevated | 16 |
| 26 | MLPA | 36 | Presymptomatic | Yes | No | No | Elevated | 18 |
| 27 | MLPA | 39 | Presymptomatic | Yes | No | No | Elevated | 18 |
| 28 | MLPA | 40 | Mild | Yes | na | No | Elevated | 13 |
| 29 | MLPA | 40 | Mild | Yes | na | No | Elevated | 13 |
| 30 | MLPA | 40 | Mild | Yes | No | No | Elevated | 13 |
| 31 | MLPA | 46 | Mild | Yes | na | No | Elevated | 13 |
| 32 | Del/dup test | 47 | na | Yes | na | na | na | CNDR |
| 33 | MLPA | 47 | Mild | Yes | na | No | na | 17 |
| 34 | MLPA | 49 | Presymptomatic | Yes | No | No | Elevated | 18 |
| 35 | mPCR & | 49 | Mild | Yes | Yes | na | 1300 | 11 |
| | Southern | | | | | | | |
| | blot | | | | | | | |
| 36 | MLPA | 50 | Mild | Yes | na | No | na | 17 |
| 37 | MLPA | 50 | Mild | Yes | na | No | Elevated | 13 |
| 38 | MLPA | 53 | Mild | Yes | No | No | na | 17 |
| 39 | MLPA | 54 | Mild | Yes | Yes | No | Elevated | 13 |
| 40 | MLPA | 55 | Mild | Yes | na | No | Elevated | 13 |
| 41 | Del/dup test | 58 | na | Yes | No | na | na | CNDR |
| 42 | MLPA | 61 | Mild | Yes | No | No | na | 17 |
| 43 | MLPA | 62 | Presymptomatic | Yes | No | No | Elevated | 18 |
| 44 | MLPA | 63 | Asymptomatic | Yes | Yes | No | Elevated | 13 |
| 45 | Del/dup test | 65 | na | Yes | No | na | na | CNDR |
| 46 | MLPA | 66 | Presymptomatic | Yes | No | No | Elevated | 18 |
| 47 | MLPA | 69 | Asymptomatic | Yes | No | na | 854 | 14 |
| 48 | MLPA | 76 | Mild | Yes | na | na | Elevated | 16 |
| 49 | mPCR & | 87 | Mild | Yes | Yes | na | 670 | 11 |
| | Southern | | | by 79 yrs | | | | |
| | blot | | | | | | | |

MLPA, multiplex ligation-dependent probe amplification; Del/dup test, deletion and duplication testing; mPCR, multiplex PCR;
^{a}, severity in accordance with the criteria of the authors;
na, not available.

TABLE 4
Prediction of non-specific binding sites of AO sequences in a human genome.

| | No. of | | No. of | | No. of |
|---|---|---|---|---|---|
| | untargeted | | untargeted | | untargeted |
| Cocktail set no. 1 | sites | Cocktail set no. 2 | sites | Cocktail set no. 3 | sites |
| Ex45_Ac9_30mer | 2 | hEx45_Ac4_25mer | 92 | Ex45_Ac9_30mer | 2 |
| Ex46_Ac52_30mer | 19 | hEx46_Ac103_25mer | 256 | Ex46_Ac93_30mer | 3 |
| Ex47_Ac50_30mer | 3 | hEx47_Ac21_25mer | 45 | Ex47_Ac13_30mer | 3 |
| Ex48_Ac7_30mer | 28 | hEx48_Ac-2_25mer | 144 | Ex48_Ac7_30mer | 28 |
| Ex49_Ac17_30mer | 4 | hEx49_Ac23_25mer | 70 | Ex48_Ac78_30mer | 13 |
| Ex50_Ac19_30mer | 0 | hEx50_Ac47_25mer | 226 | Ex49_Ac17_30mer | 4 |
| Ex51_Ac5_30mer | 3 | hEx51_Ac65_25mer | 282 | Ex50_Ac19_30mer | 0 |
| Ex52_Ac24_30mer | 1 | hEx52_Ac3_25mer | 135 | Ex51_Ac0_30mer | 7 |
| Ex53_Ac9_30mer | 14 | hEx53_Ac43_25mer | 40 | Ex52_Ac24_30mer | 1 |
| Ex54_Ac42_30mer | 5 | hEx54_Ac22_25mer | 180 | Ex53_Ac26_30mer | 5 |
| Ex55_Ac0_30mer | 20 | hEx55_Ac83_25mer | 179 | Ex54_Ac42_30mer | 5 |
| | | | | Ex55_Ac0_30mer | 20 |
| Total | 99 | | 1649 | | 91 |

Untargeted sites indicate the genome sites predicted by the GGGenome of which nucleotide sequences differ in 5 and 4 nucleotides with mismatches/gaps from 30-mer and 25-mer AO sequences, respectively.
AcXX, distance from an acceptor splice site.

TABLE 5
RT-PCR primers used in this study.

| ID | Name | Sequence (5′ to 3′) | Amplicon size | SEQ ID NO: |
|---|---|---|---|---|
| 1F | Ex43/44_167- | GACAAGGGCGATTTGACAG | 309 bp in ex45-55 skipping | 1 |
| | 12_hDMD_F | | | |
| 1R | Ex56_135- | TCCGAAGTTCACTCCACTTG | | 2 |
| | 154_hDMD_R | | | |
| 2R | Ex46_63-83_hDMD_R | TGTTATCTGCTTCCTCCAACC | 238 bp in ex45 skipping with 1F | 3 |
| 3F | Ex45_47-65_hDMD_F | TGAATGCAACTGGGGAAGA | 208 bp in ex46 skipping | 4 |
| 3R | Ex47_59-78_hDMD_R | ACTTACAAGCACGGGTCCTC | | 5 |
| 4F | Ex46_103- | ACCTGGAAAAGAGCAGCAAC | 173 bp in ex47 skipping | 6 |
| | 122_hDMD_F | | | |
| 4R | Ex48_106- | TAGGAGATAACCACAGCAGCAG | | 7 |
| | 127_hDMD_R | | | |
| 5F | Ex47_63-82_hDMD_F | ACCCGTGCTTGTAAGTGCTC | 232 bp in ex48 skipping | 8 |
| 5R | Ex50_23-42_hDMD_R | GTTTACCGCCTTCCACTCAG | 316 bp in ex49 skipping | 9 |
| 6F | Ex48_153- | CCAACCAAACCAAGAAGGAC | 232 bp in ex50 skipping | 10 |
| | 172_hDMD_F | | | |
| 6R | Ex51_76-96_hDMD_R | CCTCCAACATCAAGGAAGATG | | 11 |
| 7F | Ex49/50_94- | CAGCCAGTGAAGAGGAAGTTAG | 220 bp in ex51 skipping for ex52 del. | 12 |
| | 10_hDMD_F | | | |
| 7R | Ex53_80-99_hDMD_R | CCAGCCATTGTGTTGAATCC | | 13 |
| 8F | Ex51_188- | GGTGGGTGACCTTGAGGATA | 402 bp in ex52 skipping | 14 |
| | 207_hDMD_F | | | |
| 8R | Ex54_125- | GCTTCTCCAAGAGGCATTGA | 190 bp in ex53 skipping for ex52 del. | 15 |
| | 144_hDMD_R | | | |
| 9F | Ex53_93-112_hDMD_F | TGGCTGGAAGCTAAGGAAGA | 242 bp in ex54 skipping | 16 |
| 9R | Ex55_102- | CCTGTAGGACATTGGCAGTTG | | 17 |
| | 122_hDMD_R | | | |
| 10F | Ex54_48-67_hDMD_F | AAATGACTTGGCCCTGAAAC | 212 bp in ex55 skipping | 18 |
| 10R | Ex56_86-104_hDMD_R | AGGACTGCATCATCGGAAC | | 19 |
| 11F | hGAPDH_662-81_Fwd1 | TCCCTGAGCTGAACGGGAAG | 218 bp | 20 |
| 11R | hGAPDH_860-79_Rv1 | GGAGGAGTGGGTGTCGCTGT | | 21 |
| 12F | mGapdh_999-1015_Fwd | GCTCATTTCCTGGTATG | 93 bp | 22 |
| 12R | mGapdh_1075-91_Rv | TCCAGGGTTTCTTACTC | | 23 |

TABLE 6
Sequences of PMOs used in FIG. 2, FIG. 13, and FIG. 14

| | | | Target | SEQ ID |
|---|---|---|---|---|
| Oligo Name | Sequence | Length | exon | NO. |
| Ac2 | GTTTGCCGCTGCCCAATGCCATCCTGGAGT | 30 | 45 | 24 |
| Ac9_Exon 45 | GACAACAGTTTGCCGCTGCCCAATGCCATC | 30 | 45 | 25 |
| hAc4 | TGCCGCTGCCCAATGCCATCCTGGA | 25 | 45 | 26 |
| Ac-2 | GCCGCTGCCCAATGCCATCCTGGAGTTCCT | 30 | 45 | 27 |
| Ac54 | TGAGGATTGCTGAATTATTTCTTCCCCAGT | 30 | 45 | 28 |
| Ac40 | TTATTTCTTCCCCAGTTGCATTCAATGTTC | 30 | 45 | 29 |
| Ac52 | GTTATCTGCTTCCTCCAACCATAAAACAAA | 30 | 46 | 30 |
| hAc103 | CTTTTAGTTGCTGCTCTTTTCCAGG | 25 | 46 | 31 |
| Ac89 | GCTGCTCTTTTCCAGGTTCAAGTGGGATAC | 30 | 46 | 32 |
| Ac93 | AGTTGCTGCTCTTTTCCAGGTTCAAGTGGG | 30 | 46 | 33 |
| Ac79 | TCCAGGTTCAAGTGGGATACTAGCAATGTT | 30 | 46 | 34 |
| Ac4 | TTCCCTGGCGCAGGGGCAACTCTTCCACCA | 30 | 47 | 35 |
| Ac50 | GCACTTACAAGCACGGGTCCTCCAGTTTCA | 30 | 47 | 36 |
| hAc21 | ATTGTTTGAGAATTCCCTGGCGCAG | 25 | 47 | 37 |
| Ac-18 | TTCCACCAGTAACTGAAACAGACAAATGCA | 30 | 47 | 38 |
| Ac-9_Exon | GGGCAACTCTTCCACCAGTAACTGAAACAG | 30 | 47 | 39 |
| 47 | | | | |
| Ac59 | CTTATGGGAGCACTTACAAGCACGGGTCCT | 30 | 47 | 40 |
| Ac13 | GTTTGAGAATTCCCTGGCGCAGGGGCAACT | 30 | 47 | 41 |
| Ac3 | TTCTCCTTGTTTCTCAGGTAAAGCTCTGGA | 30 | 48 | 42 |
| Ac7 | CAATTTCTCCTTGTTTCTCAGGTAAAGCTC | 30 | 48 | 43 |
| hAc-2 | TTCTCAGGTAAAGCTCTGGAAACCT | 25 | 48 | 44 |
| Ac39 | TTCAAGCTGCCCAAGGTCTTTTATTTGAGC | 30 | 48 | 45 |
| Ac78 | CAGATGATTTAACTGCTCTTCAAGGTCTTC | 30 | 48 | 46 |
| Ac17 | ATCTCTTCCACATCCGGTTGTTTAGCTTGA | 30 | 49 | 47 |
| hAc23 | AATCTCTTCCACATCCGGTTGTTTA | 25 | 49 | 48 |
| Ac31 | GCCCTTTAGACAAAATCTCTTCCACATCCG | 30 | 49 | 49 |
| Ac74 | CACTGGCTGAGTGGCTGGTTTTTCC | 25 | 49 | 50 |
| Ac63 | CCACTCAGAGCTCAGATCTTCTAACTTCCT | 30 | 50 | 51 |
| Ac19 | ACGGTTTACCGCCTTCCACTCAGAGCTCAG | 30 | 50 | 52 |
| hAc47 | CTGCTTTGCCCTCAGCTCTTGAAGT | 25 | 50 | 53 |
| Ac0 | GTGTCACCAGAGTAACAGTCTGAGTAGGAG | 30 | 51 | 54 |
| Ac5 | AGGTTGTGTCACCAGAGTAACAGTCTGAGT | 30 | 51 | 55 |
| Ac65:Ete | CTCCAACATCAAGGAAGATGGCATTTCTAG | 30 | 51 | 56 |
| hAc65 | ACATCAAGGAAGATGGCATTTCTAG | 25 | 51 | 57 |
| Ad 1 | ACGCCTCTGTTCCAAATCCTGCATTGTTGC | 30 | 52 | 58 |
| Ac24 | CCAACTGGGGACGCCTCTGTTCCAAATCCT | 30 | 52 | 59 |
| hAc3 | GCCTCTGTTCCAAATCCTGCATTGT | 25 | 52 | 60 |
| Ac26 | CCTCCGGTTCTGAAGGTGTTCTTGTACTTC | 30 | 53 | 61 |
| Ac9_Exon 53 | GTTCTTGTACTTCATCCCACTGATTCTGAA | 30 | 53 | 62 |
| hAc43 | ATTCAACTGTTGCCTCCGGTTCTGA | 25 | 53 | 63 |
| Ac42 | GAGAAGTTTCAGGGCCAAGTCATTTGCCAC | 30 | 54 | 64 |
| hAc22 | GCCACATCTACATTTGTCTGCCACT | 25 | 54 | 65 |
| Ac0_Exon 55 | TCTTCCAAAGCAGCCTCTCGCTCACTCACC | 30 | 55 | 66 |
| hAc83 | GCAGTTGTTTCAGCTTCTGTAAGCC | 25 | 55 | 67 |
| Ac61 | ACTAGCAATGTTATCTGCTTCCTCCAACCA | 30 | 46 | 68 |
| Ac21 | TCATTTAAATCTCTTTGAAATTCTGACAAG | 30 | 46 | 69 |
| Ac119 | CCTTGACTTGCTCAAGCTTTTCTTTTAGTT | 30 | 46 | 70 |
| Ac5_Exon 50 | GCCTTCCACTCAGAGCTCAGATCTTCTAAC | 30 | 50 | 71 |
| Ac68 | GTGGTCAGTCCAGGAGCTAGGTCAGGCTGC | 30 | 50 | 72 |
| Ac35 | GCCCTCAGCTCTTGAAGTAAACGGTTTACC | 30 | 50 | 73 |

TABLE 7
Compositions of the minimized exon
45-55 skipping PMO cocktails.

| Cocktail name | DMD exons targeted | Total # PMOs* |
|---|---|---|
| all | 45, 46, 47, 48, 49, | 11 |
| | 50, 51, 52, 53, 54, 55 | |
| base | 45, 47, 49, 51, 53, 55 | 6 |
| base-51 | 45, 47, 49, 53, 55 | 5 |
| block | 45, 49, 50, 52, 53, 55 | 6 |
| 3-PMO | 45, 50, 55 | 3 |

*PMOs used for each exon are: 45, Ac9; 46, Ac93; 47, Ac13; 48, Ac7 and Ac78; 49, Ac17; 50, Ac19; 51, Ac0; 52, Ac24; 53, Ac26; 54, Ac42; 55, Ac0

### REFERENCES

1. Koenig, M, Hoffman, E P, Bertelson, C J, Monaco, A P, Feener, C, and Kunkel, L M (1987). Complete cloning of the Duchenne muscular dystrophy (DMD) cDNA and preliminary genomic organization of the DMD gene in normal and affected individuals. Cell 50: 509-517.
2. Echigoya, Y, Lim, KRQ, Nakamura, A, and Yokota, T (2018). Multiple Exon Skipping in the Duchenne Muscular Dystrophy Hot Spots: Prospects and Challenges. J Pers Med 8: 41.
3. Bladen, C L, Salgado, D, Monges, S, Foncuberta, M E, Kekou, K, Kosma, K, et al. (2015). The TREAT-NMD DMD Global Database: analysis of more than 7,000 Duchenne muscular dystrophy mutations. Hum Mutat 36: 395-402.
4. Monaco, A P, Bertelson, C J, Liechti-Gallati, S, Moser, H, and Kunkel, L M (1988).

An explanation for the phenotypic differences between patients bearing partial deletions of the DMD locus. Genomics 2: 90-95.
5. Lim, K R, Maruyama, R, and Yokota, T (2017). Eteplirsen in the treatment of Duchenne muscular dystrophy. Drug Des Devel Ther 11: 533-545.
6. Rinaldi, C, and Wood, M J A (2018). Antisense oligonucleotides: the next frontier for treatment of neurological disorders. Nat Rev Neurol 14: 9-21.
7. Komaki, H, Nagata, T, Saito, T, Masuda, S, Takeshita, E, Sasaki, M, et al. (2018). Systemic administration of the antisense oligonucleotide NS-065/NCNP-01 for skipping of exon 53 in patients with Duchenne muscular dystrophy. Sci Transl Med 10.
8. Echigoya, Y, and Yokota, T (2014). Skipping Multiple Exons of Dystrophin Transcripts Using Cocktail Antisense Oligonucleotides. Nucleic Acid Ther 24: 57-68.
9. van den Bergen, J C, Wokke, B H, Janson, A A, van Duinen, S G, Hulsker, M A, Ginjaar, H B, et al. (2013). Dystrophin levels and clinical severity in Becker muscular dystrophy patients. J Neurol Neurosurg Psychiatry.
10. Bello, L, Campadello, P, Barp, A, Fanin, M, Semplicini, C, Soraru, G, et al. (2016). Functional changes in Becker muscular dystrophy: implications for clinical trials in dystrophinopathies. Sci Rep 6: 32439.
11. Nakamura, A, Shiba, N, Miyazaki, D, Nishizawa, H, Inaba, Y, Fueki, N, et al. (2016). Comparison of the phenotypes of patients harboring in-frame deletions starting at exon 45 in the Duchenne muscular dystrophy gene indicates potential for the development of exon skipping therapy. J Hum Genet.
12. Mori-Yoshimura, M, Mitsuhashi, S, Nakamura, H, Komaki, H, Goto, K, Yonemoto, N, et al. (2018). Characteristics of Japanese Patients with Becker Muscular Dystrophy and Intermediate Muscular Dystrophy in a Japanese National Registry of Muscular Dystrophy (Remudy): Heterogeneity and Clinical Variation. J Neuromuscul Dis 5: 193-203.
13. Beroud, C, Tuffery-Giraud, S, Matsuo, M, Hamroun, D, Humbertclaude, V, Monnier, N, et al. (2007). Multiexon skipping leading to an artificial DMD protein lacking amino acids from exons 45 through 55 could rescue up to 63% of patients with Duchenne muscular dystrophy. Hum Mutat 28: 196-202.
14. Ferreiro, V, Giliberto, F, Muniz, G M, Francipane, L, Marzese, DM, Mampel, A, et al. (2009). Asymptomatic Becker muscular dystrophy in a family with a multiexon deletion. Muscle Nerve 39: 239-243.
15. Nakamura, A, Yoshida, K, Fukushima, K, Ueda, H, Urasawa, N, Koyama, J, et al. (2008). Follow-up of three patients with a large in-frame deletion of exons 45-55 in the Duchenne muscular dystrophy (DMD) gene. J Clin Neurosci 15: 757-763.
16. Anthony, K, Cirak, S, Torelli, S, Tasca, G, Feng, L, Arechavala-Gomeza, V, et al. (2011). Dystrophin quantification and clinical correlations in Becker muscular dystrophy: implications for clinical trials. Brain 134: 3547-3559.
17. van den Bergen, J C, Schade van Westrum, S M, Dekker, L, van der Kooi, A J, de Visser, M, Wokke, B H, et al. (2014). Clinical characterisation of Becker muscular dystrophy patients predicts favourable outcome in exon-skipping therapy. J Neurol Neurosurg Psychiatry 85: 92-98.
18. Taglia, A, Petillo, R, D'Ambrosio, P, Picillo, E, Torella, A, Orsini, C, et al. (2015). Clinical features of patients with dystrophinopathy sharing the 45-55 exon deletion of DMD gene. Acta Myol 34: 9-13.
19. van Vliet, L, de Winter, C L, van Deutekom, J C, van Ommen, G J, and Aartsma-Rus, A (2008). Assessment of the feasibility of exon 45-55 multiexon skipping for Duchenne muscular dystrophy. BMC Med Genet 9: 105.
20. Aoki, Y, Yokota, T, Nagata, T, Nakamura, A, Tanihata, J, Saito, T, et al. (2012). Bodywide skipping of exons 45-55 in dystrophic mdx52 mice by systemic antisense delivery. Proc Natl Acad Sci USA 109: 13763-13768.
21. Lee, J, Echigoya, Y, Duddy, W, Saito, T, Aoki, Y, Takeda, S, et al. (2018). Antisense PMO cocktails effectively skip dystrophin exons 45-55 in myotubes transdifferentiated from DMD patient fibroblasts. PLoS One 13: e0197084.
22. Echigoya, Y, Mouly, V, Garcia, L, Yokota, T, and Duddy, W (2015). In silico screening based on predictive algorithms as a design tool for exon skipping oligonucleotides in duchenne muscular dystrophy. PLoS One 10: e0120058.
23. Anthony, K, Arechavala-Gomeza, V, Ricotti, V, Torelli, S, Feng, L, Janghra, N, et al. (2014). Biochemical characterization of patients with in-frame or out-of-frame DMD deletions pertinent to exon 44 or 45 skipping. JAMA Neurol 71: 32-40.
24. Echigoya, Y, Aoki, Y, Miskew, B, Panesar, D, Touznik, A, Nagata, T, et al. (2015). Long-term efficacy of systemic multiexon skipping targeting dystrophin exons 45-55 with a cocktail of vivo-morpholinos in mdx52 mice. Mol Ther Nucleic Acids 4: e225.
25. Echigoya, Y, Lim, K R Q, Trieu, N, Bao, B, Miskew Nichols, B, Vila, M C, et al. (2017). Quantitative Antisense Screening and Optimization for Exon 51 Skipping in Duchenne Muscular Dystrophy. Mol Ther 25: 2561-2572.
26. Ferguson, D P, Dangott, L J, and Lightfoot, J T (2014). Lessons learned from vivo-morpholinos: How to avoid vivo-morpholino toxicity. Biotechniques 56: 251-256.
27. Krtkova, J, and Paredez, AR (2017). Use of Translation Blocking Morpholinos for Gene Knockdown in Giardia lamblia. Methods Mol Biol 1565: 123-140.
28. Chow, G, Morcos, P A, and Moulton, H M (2017). Aggregation and Disaggregation of Morpholino Oligomers in Solution. Methods Mol Biol 1565: 31-38.
29. Suzuki, H, Aoki, Y, Kameyama, T, Saito, T, Masuda, S, Tanihata, J, et al. (2016). Endogenous Multiple Exon Skipping and Back-Splicing at the DMD Mutation Hotspot. Int J Mol Sci 17.
30. Tanihata, J, Nagata, T, Ito, N, Saito, T, Nakamura, A, Minamisawa, S, et al. (2018).

Truncated dystrophin ameliorates the dystrophic phenotype of mdx mice by reducing sarcolipin-mediated SERCA inhibition. Biochemical and Biophysical Research Communications.
31. Moulton, H M, and Moulton, J D (2010). Morpholinos and their peptide conjugates: therapeutic promise and challenge for Duchenne muscular dystrophy. Biochim Biophys Acta 1798: 2296-2303.
32. Yokota, T, Nakamura, A, Nagata, T, Saito, T, Kobayashi, M, Aoki, Y, et al. (2012). Extensive and prolonged restoration of dystrophin expression with vivo-morpholino-mediated multiple exon skipping in dystrophic dogs. Nucleic Acid Ther 22: 306-315.
33. Lim, K R Q, Echigoya, Y, Nagata, T, Kuraoka, M, Kobayashi, M, Aoki, Y, et al. (2019). Efficacy of multi-exon skipping treatment in Duchenne muscular dystrophy dog model neonates. Mol Ther 27: 76-86.
34. Echigoya, Y, Nakamura, A, Nagata, T, Urasawa, N, Lim, K R, Trieu, N, et al. (2017). Effects of systemic multiexon skipping with peptide-conjugated morpholinos in the heart of a dog model of Duchenne muscular dystrophy. Proc Natl Acad Sci USA 114: 4213-4218.
35. Aartsma-Rus, A, and Arechavala-Gomeza, V (2018). Why dystrophin quantification is key in the eteplirsen saga. Nat Rev Neurol.
36. Young, C S, Mokhonova, E, Quinonez, M, Pyle, A D, and Spencer, M J (2017). Creation of a Novel Humanized Dystrophic Mouse Model of Duchenne Muscular Dystrophy and Application of a CRISPR/Cas9 Gene Editing Therapy. J Neuromuscul Dis 4: 139-145.
37. Veltrop, M, van Vliet, L, Hulsker, M, Claassens, J, Brouwers, C, Breukel, C, et al. (2018). A dystrophic Duchenne mouse model for testing human antisense oligonucleotides. PLoS One 13: e0193289.
38. Kaspar, R W, Allen, H D, Ray, W C, Alvarez, C E, Kissel, J T, Pestronk, A, et al. (2009). Analysis of dystrophin deletion mutations predicts age of cardiomyopathy onset in becker muscular dystrophy. Circ Cardiovasc Genet 2: 544-551.
39. Dombernowsky, N W, Olmestig, J N E, Witting, N, and Kruuse, C (2018). Role of neuronal nitric oxide synthase (nNOS) in Duchenne and Becker muscular dystrophies—Still a possible treatment modality? Neuromuscul Disord 28: 914-926.
40. Gentil, C, Leturcq, F, Ben Yaou, R, Kaplan, J C, Laforet, P, Penisson-Besnier, I, et al. (2012). Variable phenotype of de145-55 Becker patients correlated with nNOSmu mislocalization and RYR1 hypernitrosylation. Hum Mol Genet 21: 3449-3460.
41. Molza, A E, Mangat, K, Le Rumeur, E, Hubert, J F, Menhart, N, and Delalande, O (2015). Structural Basis of Neuronal Nitric-oxide Synthase Interaction with Dystrophin Repeats 16 and 17. J Biol Chem 290: 29531-29541.
42. Amann, K J, Guo, A W, and Ervasti, J M (1999). Utrophin lacks the rod domain actin binding activity of dystrophin. J Biol Chem 274: 35375-35380.
43. Legardinier, S, Raguenes-Nicol, C, Tascon, C, Rocher, C, Hardy, S, Hubert, J F, et al. (2009). Mapping of the lipid-binding and stability properties of the central rod domain of human dystrophin. J Mol Biol 389: 546-558.
44. Nicolas, A, Raguenes-Nicol, C, Ben Yaou, R, Ameziane-Le Hir, S, Cheron, A, Vie, V, et al. (2014). Becker muscular dystrophy severity is linked to the structure of dystrophin. Hum Mol Genet.
45. U.S. Food and Drug Administration (2013). Guidance for Industry: Codevelopment of Two or More New Investigational Drugs for Use in Combination. https://www.fda.gov/downloads/Drugs/GuidanceComplianceRegulatoryInformatio n/Guidances/UCM236669.pdf.
46. Yokota, T, Duddy, W, Echigoya, Y, and Kolski, H (2012). Exon skipping for nonsense mutations in Duchenne muscular dystrophy: too many mutations, too few patients? Expert Opin Biol Ther 12: 1141-1152.
47. Aartsma-Rus, A, Fokkema, I, Verschuuren, J, Ginjaar, I, van Deutekom, J, van Ommen, GJ, et al. (2009). Theoretic applicability of antisense-mediated exon skipping for Duchenne muscular dystrophy mutations. Hum Mutat 30: 293-299.
48. Lee, J J A, Maruyama, R, Duddy, W, Sakurai, H, and Yokota, T (2018). Identification of Novel Antisense-Mediated Exon Skipping Targets in DYSF for Therapeutic Treatment of Dysferlinopathy. Mol Ther Nucleic Acids 13: 596-604.
49. Eisen, J S, and Smith, J C (2008). Controlling morpholino experiments: don't stop making antisense. Development 135: 1735-1743.
50. Mamchaoui, K, Trollet, C, Bigot, A, Negroni, E, Chaouch, S, Wolff, A, et al. (2011). Immortalized pathological human myoblasts: towards a universal tool for the study of neuromuscular disorders. Skelet Muscle 1: 34. 51. Bremmer-Bout, M, Aartsma-Rus, A, de Meijer, E J, Kaman, W E, Janson, A A, Vossen, R H, et al. (2004). Targeted exon skipping in transgenic hDMD mice: A model for direct preclinical screening of human-specific antisense oligonucleotides. Mol Ther 10: 232-240.
52. Kudoh, H, Ikeda, H, Kakitani, M, Ueda, A, Hayasaka, M, Tomizuka, K, et al. (2005). Anew model mouse for Duchenne muscular dystrophy produced by 2.4 Mb deletion of dystrophin gene using Cre-loxP recombination system. Biochem Biophys Res Commun 328: 507-516.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

what is claimed is:

1. An antisense oligonucleotide capable of binding to exon 46 of human dystrophin pre-mRNA, wherein binding of the antisense oligonucleotide takes place entirely within the region between +89 and +149 of the pre-mRNA sequence, and wherein the antisense oligonucleotide comprises at least 26 base pairs.

2. The antisense oligonucleotide of claim 1, wherein the antisense oligonucleotide comprises at least 27, at least 28 bases, at least 29 bases, or at least 30 bases.

3. The antisense oligonucleotide according to claim 1 or 2, wherein the antisense oligonucleotide consists of 30 bases.

4. The antisense oligonucleotide according to any one of claims 1 to 3, wherein the antisense oligonucleotide is at least 70%, at least 80%, at least 90%, at least 95%, at least 96%, at least 97%, at least 98% at least 99% complementary to a sequence of exon 46 of human dystrophin pre-mRNA falling within the region.

5. The antisense oligonucleotide according to any one of claims 1 to 4, wherein the antisense oligonucleotide is hybridisable to a sequence of exon 46 of human dystrophin pre-mRNA falling within the region.

6. The antisense oligonucleotide according to any one of claims 1 to 5, wherein the antisense oligonucleotide comprises at least 26 bases of one of the following sequences Ac89 (SEQ ID NO. 32), Ac93 (SEQ ID NO. 33), or Ac119 (SEQ ID NO. 70).

7. An antisense oligonucleotide capable of binding to exon 46 of human dystrophin pre-mRNA, wherein binding of the antisense oligonucleotide takes place entirely within the region between +89 and +149 of the pre-mRNA sequence, and wherein the antisense oligonucleotide comprises at least 25 base pairs, wherein the antisense oligonucleotide comprises the sequence hAc103 (SEQ ID NO. 31).

8. An antisense oligonucleotide capable of binding to exon 50 of human dystrophin pre-mRNA, wherein binding of the antisense oligonucleotide takes place entirely within the region between +5 and +98 of the pre-mRNA sequence, and wherein the antisense oligonucleotide comprises at least 26 base pairs.

9. The antisense oligonucleotide of claim 8, wherein the antisense oligonucleotide comprises at least 27, at least 28 bases, at least 29 bases, or at least 30 bases.

10. The antisense oligonucleotide according to claim 8 or 9, wherein the antisense oligonucleotide consists of 30 bases.

11. The antisense oligonucleotide according to any one of claims 8 to 10, wherein the antisense oligonucleotide is at least 70%, at least 80%, at least 90%, at least 95%, at least 96%, at least 97%, at least 98% at least 99% complementary to a sequence of exon 50 of human dystrophin pre-mRNA falling within the region.

12. The antisense oligonucleotide according to any one of claims 8 to 11, wherein the antisense oligonucleotide is hybridisable to a sequence of exon 50 of human dystrophin pre-mRNA falling within the region.

13. The antisense oligonucleotide according to any one of claims 8 to 12, wherein the antisense oligonucleotide comprises at least 26 bases of one of the following sequences Ac5 (SEQ ID NO. 71), Ac19 (SEQ ID NO. 52), Ac63 (SEQ ID NO. 51), or Ac68 (SEQ ID NO. 72).

14. An antisense cocktail containing 3 or more antisense oligonucleotides from Set no. 1, Set no. 2, or Set no. 3.

15. The antisense cocktail of claim 14, wherein the antisense oligonucleotides from Set no. 1, Set no. 2, or Set no. 3, is at least 70%, at least 80%, at least 90%, at least 95%, at least 96%, at least 97%, at least 98% at least 99% complementary to the antisense oligonucleotides from Set no. 1, Set no. 2, or Set no. 3.

16. A conjugate comprising an antisense oligonucleotide according to any one of claims 1 to 13 and a carrier, wherein the carrier is conjugated to the antisense oligonucleotide.

17. A conjugate according to claim 16, wherein the carrier is operable to transport the antisense oligonucleotide into a target cell.

18. A conjugate according to claim 16 or 17, wherein the carrier is selected from a peptide, a small molecule chemical, a polymer, a nanoparticle, a lipid, a liposome or an exosome.

19. A conjugate according to any one of claims 16 to 18, wherein the carrier is a cell penetrating peptide.

20. A conjugate according to any one of claims 16 to 19, wherein the carrier is an arginine-rich cell penetrating peptide.

21. A cell loaded with a conjugate of any one of claims 16 to 20.

22. A pharmaceutical composition comprising an antisense oligonucleotide according to any one of claims 1 to 15, and/or a conjugate according to any one of claims 16 to 21, and a pharmaceutically acceptable excipient.

23. An antisense oligonucleotide of any one of claims 1 to 15, for use in the treatment of a muscular disorder in a subject.

24. A conjugate of any one of claims 1 to 15, for use in the treatment of a muscular disorder in a subject.

25. The antisense oligonucleotide for use according to claim 23 or the conjugate of claim 24, wherein the muscular disorder is a disorder resulting from a genetic mutation in a gene associated with muscle function.

26. The antisense oligonucleotide for use according to claim 23 or the conjugate of claim 24, wherein the muscular disorder is Duchenne muscular dystrophy or Becker muscular dystrophy.


SEQUENCE LISTING (77 sequences)

SEQ ID NO: 1 - DNA, 19 bp, Artificial Sequence
  Ex43/44_167-12_hDMD_F
gacaagggcgatttgacag

SEQ ID NO: 2 - DNA, 20 bp, Artificial Sequence
  Ex56_135-154_hDMD_R
tccgaagttcactccacttg

SEQ ID NO: 3 - DNA, 21 bp, Artificial Sequence
  Ex46_63-83_hDMD_R
tgttatctgcttcctccaacc

SEQ ID NO: 4 - DNA, 19 bp, Artificial Sequence
  Ex45_47-65_hDMD_F
tgaatgcaactggggaaga

SEQ ID NO: 5 - DNA, 20 bp, Artificial Sequence
  Ex47_59-78_hDMD_R
acttacaagcacgggtcctc

SEQ ID NO: 6 - DNA, 20 bp, Artificial Sequence
  Ex46_103-122_hDMD_F
acctggaaaagagcagcaac

SEQ ID NO: 7 - DNA, 22 bp, Artificial Sequence
  Ex48_106-127_hDMD_R
taggagataaccacagcagcag

SEQ ID NO: 8 - DNA, 20 bp, Artificial Sequence
  Ex47_63-82_hDMD_F
acccgtgcttgtaagtgctc

SEQ ID NO: 9 - DNA, 20 bp, Artificial Sequence
  Ex50_23-42_hDMD_R
gtttaccgccttccactcag

SEQ ID NO: 10 - DNA, 20 bp, Artificial Sequence
  Ex48_153-172_hDMD_F
ccaaccaaaccaagaaggac

SEQ ID NO: 11 - DNA, 21 bp, Artificial Sequence
  Ex51_76-96_hDMD_R
cctccaacatcaaggaagatg

SEQ ID NO: 12 - DNA, 22 bp, Artificial Sequence
  Ex49/50_94-10_hDMD_F
cagccagtgaagaggaagttag

SEQ ID NO: 13 - DNA, 20 bp, Artificial Sequence
  Ex53_80-99_hDMD_R
ccagccattgtgttgaatcc

SEQ ID NO: 14 - DNA, 20 bp, Artificial Sequence
  Ex51_188-207_hDMD_F
ggtgggtgaccttgaggata

SEQ ID NO: 15 - DNA, 20 bp, Artificial Sequence
  Ex54_125-144_hDMD_R
gcttctccaagaggcattga

SEQ ID NO: 16 - DNA, 20 bp, Artificial Sequence
  Ex53_93-112_hDMD_F
tggctggaagctaaggaaga

SEQ ID NO: 17 - DNA, 21 bp, Artificial Sequence
  Ex55_102-122_hDMD_R
cctgtaggacattggcagttg

SEQ ID NO: 18 - DNA, 20 bp, Artificial Sequence
  Ex54_48-67_hDMD_F
aaatgacttggccctgaaac

SEQ ID NO: 19 - DNA, 19 bp, Artificial Sequence
  Ex56_86-104_hDMD_R
aggactgcatcatcggaac

SEQ ID NO: 20 - DNA, 20 bp, Artificial Sequence
  hGAPDH_662-81_Fwd1
tccctgagctgaacgggaag

SEQ ID NO: 21 - DNA, 20 bp, Artificial Sequence
  hGAPDH_860-79_Rv1
ggaggagtgggtgtcgctgt

SEQ ID NO: 22 - DNA, 17 bp, Artificial Sequence
  mGapdh_999-1015_Fwd
gctcatttcctggtatg

SEQ ID NO: 23 - DNA, 17 bp, Artificial Sequence
  mGapdh_1075-91_Rv
tccagggtttcttactc

SEQ ID NO: 24 - DNA, 30 bp, Artificial Sequence
  Ac2
gtttgccgctgcccaatgccatcctggagt

SEQ ID NO: 25 - DNA, 30 bp, Artificial Sequence
  Ac9
gacaacagtttgccgctgcccaatgccatc

SEQ ID NO: 26 - DNA, 25 bp, Artificial Sequence
  hAc4
tgccgctgcccaatgccatcctgga

SEQ ID NO: 27 - DNA, 30 bp, Artificial Sequence
  Ac-2
gccgctgcccaatgccatcctggagttcct

SEQ ID NO: 28 - DNA, 30 bp, Artificial Sequence
  Ac54
tgaggattgctgaattatttcttccccagt

SEQ ID NO: 29 - DNA, 30 bp, Artificial Sequence
  Ac40
ttatttcttccccagttgcattcaatgttc

SEQ ID NO: 30 - DNA, 30 bp, Artificial Sequence
  Ac52
gttatctgcttcctccaaccataaaacaaa

SEQ ID NO: 31 - DNA, 25 bp, Artificial Sequence
  hAc103
cttttagttgctgctcttttccagg

SEQ ID NO: 32 - DNA, 30 bp, Artificial Sequence
  Ac89
gctgctcttttccaggttcaagtgggatac

SEQ ID NO: 33 - DNA, 30 bp, Artificial Sequence
  Ac93
agttgctgctcttttccaggttcaagtggg

SEQ ID NO: 34 - DNA, 30 bp, Artificial Sequence
  Ac79
tccaggttcaagtgggatactagcaatgtt

SEQ ID NO: 35 - DNA, 30 bp, Artificial Sequence
  Ac4
ttccctggcgcaggggcaactcttccacca

SEQ ID NO: 36 - DNA, 30 bp, Artificial Sequence
  Ac50
gcacttacaagcacgggtcctccagtttca

SEQ ID NO: 37 - DNA, 25 bp, Artificial Sequence
  hAc21
attgtttgagaattccctggcgcag

SEQ ID NO: 38 - DNA, 30 bp, Artificial Sequence
  Ac-18
ttccaccagtaactgaaacagacaaatgca

SEQ ID NO: 39 - DNA, 30 bp, Artificial Sequence
  Ac-9
gggcaactcttccaccagtaactgaaacag

SEQ ID NO: 40 - DNA, 30 bp, Artificial Sequence
  Ac59
cttatgggagcacttacaagcacgggtcct

SEQ ID NO: 41 - DNA, 30 bp, Artificial Sequence
  Ac13
gtttgagaattccctggcgcaggggcaact

SEQ ID NO: 42 - DNA, 30 bp, Artificial Sequence
  Ac3
ttctccttgtttctcaggtaaagctctgga

SEQ ID NO: 43 - DNA, 30 bp, Artificial Sequence
  Ac7
caatttctccttgtttctcaggtaaagctc

SEQ ID NO: 44 - DNA, 25 bp, Artificial Sequence
  hAc-2
ttctcaggtaaagctctggaaacct

SEQ ID NO: 45 - DNA, 30 bp, Artificial Sequence
  Ac39
ttcaagctgcccaaggtcttttatttgagc

SEQ ID NO: 46 - DNA, 30 bp, Artificial Sequence
  Ac78
cagatgatttaactgctcttcaaggtcttc

SEQ ID NO: 47 - DNA, 30 bp, Artificial Sequence
  Ac17
atctcttccacatccggttgtttagcttga

SEQ ID NO: 48 - DNA, 25 bp, Artificial Sequence
  hAc23
aatctcttccacatccggttgttta

SEQ ID NO: 49 - DNA, 30 bp, Artificial Sequence
  Ac31
gccctttagacaaaatctcttccacatccg

SEQ ID NO: 50 - DNA, 25 bp, Artificial Sequence
  Ac74
cactggctgagtggctggtttttcc

SEQ ID NO: 51 - DNA, 30 bp, Artificial Sequence
  Ac63
ccactcagagctcagatcttctaacttcct

SEQ ID NO: 52 - DNA, 30 bp, Artificial Sequence
  Ac19
acggtttaccgccttccactcagagctcag

SEQ ID NO: 53 - DNA, 25 bp, Artificial Sequence
  hAc47
ctgctttgccctcagctcttgaagt

SEQ ID NO: 54 - DNA, 30 bp, Artificial Sequence
  Ac0
gtgtcaccagagtaacagtctgagtaggag

SEQ ID NO: 55 - DNA, 30 bp, Artificial Sequence
  Ac5
aggttgtgtcaccagagtaacagtctgagt

SEQ ID NO: 56 - DNA, 30 bp, Artificial Sequence
  Ac65Ete
ctccaacatcaaggaagatggcatttctag

SEQ ID NO: 57 - DNA, 25 bp, Artificial Sequence
  hAc65
acatcaaggaagatggcatttctag

SEQ ID NO: 58 - DNA, 30 bp, Artificial Sequence
  Ac1
acgcctctgttccaaatcctgcattgttgc

SEQ ID NO: 59 - DNA, 30 bp, Artificial Sequence
  Ac24
ccaactggggacgcctctgttccaaatcct

SEQ ID NO: 60 - DNA, 25 bp, Artificial Sequence
  hAc3
gcctctgttccaaatcctgcattgt

SEQ ID NO: 61 - DNA, 30 bp, Artificial Sequence
  Ac26
cctccggttctgaaggtgttcttgtacttc

SEQ ID NO: 62 - DNA, 30 bp, Artificial Sequence
  Ac9_Exon 53
gttcttgtacttcatcccactgattctgaa

SEQ ID NO: 63 - DNA, 25 bp, Artificial Sequence
  hAc43
attcaactgttgcctccggttctga

SEQ ID NO: 64 - DNA, 30 bp, Artificial Sequence
  Ac42
gagaagtttcagggccaagtcatttgccac

SEQ ID NO: 65 - DNA, 25 bp, Artificial Sequence
  hAc22
gccacatctacatttgtctgccact

SEQ ID NO: 66 - DNA, 30 bp, Artificial Sequence
  Ac0_Exon 55
tcttccaaagcagcctctcgctcactcacc

SEQ ID NO: 67 - DNA, 25 bp, Artificial Sequence
  hAc83
gcagttgtttcagcttctgtaagcc

SEQ ID NO: 68 - DNA, 30 bp, Artificial Sequence
  Ac61
actagcaatgttatctgcttcctccaacca

SEQ ID NO: 69 - DNA, 30 bp, Artificial Sequence
  Ac21
tcatttaaatctctttgaaattctgacaag

SEQ ID NO: 70 - DNA, 30 bp, Artificial Sequence
  Ac119
ccttgacttgctcaagcttttcttttagtt

SEQ ID NO: 71 - DNA, 30 bp, Artificial Sequence
  Ac5_Exon 50
gccttccactcagagctcagatcttctaac

SEQ ID NO: 72 - DNA, 30 bp, Artificial Sequence
  Ac68
gtggtcagtccaggagctaggtcaggctgc

SEQ ID NO: 73 - DNA, 30 bp, Artificial Sequence
  Ac35
gccctcagctcttgaagtaaacggtttacc

SEQ ID NO: 74 - DNA, 198 bp, Artificial Sequence
  DMD exon 46
aacaattttattcttctttctccaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaaggtaattttattttctcaaatccccc

SEQ ID NO: 75 - DNA, 159 bp, Artificial Sequence
  DMD exon 50
taatgtgtatgcttttctgttaaagaggaagttagaagatctgagctctgagtggaaggcggtaaaccgtttacttcaagagctgagggcaaagcagcctgacctagctcctggactgaccactattggagcctgtaagtatactggatcccattctct

SEQ ID NO: 76 - DNA, 30 bp, Artificial Sequence
  Ex50_Ac19_30mer
gtaaacggtttaccgccttccactcagagc

SEQ ID NO: 77 - DNA, 30 bp, Artificial Sequence
  Ex52_Ac24
ggtaatgagttcttccaactggggacgcct